(19) United States
(12) Patent Application Publication
(10) Pub. No.: US 2020/0197449 A1
(43) Pub. Date: Jun. 25, 2020

(54) Compositions and Methods for Treating Alopecia and Related Disorders

(71) Applicant: Crestovo Holdings LLC, Somerville, MA (US)

(72) Inventors: Thomas J. BORODY, Five Dock (AU); Crystal Chen, Boston, MA (US)

(73) Assignee: Crestovo Holdings LLC, Somerville, MA (US)

(21) Appl. No.: 16/720,915

(22) Filed: Dec. 19, 2019

Related U.S. Application Data
(60) Provisional application No. 62/782,094, filed on Dec. 19, 2018.

(51) Int. Cl. A61K 35/74; A61P 17/14; A61K 9/00

(52) U.S. Cl. CPC A61K 35/74; A61K 9/0053; A61P 17/14

(57) ABSTRACT

The present disclosure is in the field of pharmaceutical compositions suitable for the treatment of diseases in mammals. The disclosure provides novel compositions comprising non-pathogenic fecal microbes for treating alopecia and related diseases. The disclosure also provides methods for treating a subject with the compositions disclosed herein.

### CROSS-REFERENCE TO RELATED APPLICATIONS

This application claims priority to U.S. Provisional Application No. 61/782,092, filed Dec. 19, 2018, which is hereby incorporated by reference in its entirety.

### FIELD

The present disclosure relates to pharmaceutical compositions and methods suitable for treating alopecia.

### BACKGROUND

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and increase susceptibility to pathogens, alter metabolic profiles, or induce proinflammatory signals that can lead to local or systemic inflammation or autoimmunity. Additionally, such a disrupted microbiota may be infected by incoming pathogen or pathogens, which can cause pain, diarrhea, gas, and constipation among other symptoms. Hence, the intestinal microbiota plays a significant role in the pathogenesis of many disorders such as pathogenic infections of the gut.

Implantation or administration of human colonic microbiota into the bowel of a patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE). It entails infusions through a colonoscope, an enema or via a nasojejunal or nasogastric tube of human microbiota either in the form of homogenised stool, or cultured stool components such as Clostridia, to implant in the colon and thereby displace or eradicate pathogenic bacteria, e.g., C. difficile.

Alopecia is a condition in which hair is lost from some or all parts of the body. In alopecia areata, hair loss typically occurs in patches, generally on the head. Alopecia can be mild, but in severe forms, it can spread to the whole scalp (alopecia totalis) or the whole body (alopecia universalis). Although the condition most often affects the scalp and beard, it may occur on any part of the body with hair. Onset is usually in childhood and there is no cure. Without being bound to any theory, alopecia is considered by some as an autoimmune disease where the cells of the immune system attack the hair follicle.

The lifetime risk of developing alopecia is approximately 2 percent worldwide. The disease can severely decrease quality of life, as it generally presents in childhood and early adulthood, during a time when one's peers are not experiencing hair loss and when one is at risk for bullying and exclusion. Many people with alopecia suffer severe psychological effects, including depression, anxiety, and low self-esteem/self-image.

There is no cure for alopecia, and available treatment options have shown limited success. Accordingly, there remains an unmet need for the treatment of alopecia.

### SUMMARY

The present disclosure provides compositions, methods, and dosing regimens for treating alopecia in a patient.

In one aspect, the present disclosure provides a method for treating alopecia in a subject in need thereof, where the method comprises administering to the subject a pharmaceutically active dose of a therapeutic composition comprising or derived from live non-pathogenic fecal bacteria or a sterile fecal filtrate. In one aspect, a sterile fecal filtrate originates from a donor stool. In another aspect, a sterile fecal filtrate originates from cultured microorganisms.

In another aspect, this disclosure provides use of a composition comprising live non-pathogenic fecal bacteria in the manufacture of a medication for the treatment of alopecia.

In one aspect, the present disclosure provides a method for treating alopecia in a subject in need thereof, where the method comprises administering orally to the subject a pharmaceutically active dose of a therapeutic composition comprising or derived from live, non-pathogenic, synthetic bacterial mixture or live, non-pathogenic, purified or extracted, fecal microbiota, where the dose is administered at a dosing schedule of at least once or twice daily or at least once or twice weekly for at least three, eight, ten, or twenty consecutive weeks. In another aspect, the present disclosure provides a method for treating alopecia in a subject in need thereof, where the method comprises administering orally to the subject a pharmaceutically active dose of a therapeutic composition comprising or derived from a preparation of uncultured fecal bacteria, where the dose is administered at a dosing schedule of at least once or twice daily or at least once or twice weekly for at least three, eight, ten, or twenty consecutive weeks. In a further aspect, the dose is administered at a dosing schedule of at least once or twice daily or at least once or twice weekly for at least four, five, six, seven, eleven, twelve, thirteen, fourteen, fifteen, sixteen, seventeen, eighteen, or nineteen consecutive weeks.

In one aspect, the present disclosure provides a method for treating alopecia in a subject in need thereof, where the method comprises administering orally to the subject a pharmaceutically active dose of a therapeutic composition comprising a liquid, frozen, lyophilized, or encapsulated sterile fecal filtrate, where the dose is administered at a dosing schedule of at least once or twice daily or at least once or twice weekly for at least three, eight, ten, or twenty consecutive weeks.

In one aspect, the present disclosure provides for a method for treating alopecia in a subject in need thereof, said method comprising administering to said subject an oral capsule comprising a pharmaceutically active dose of a therapeutic composition comprising live non-pathogenic fecal bacteria or a non-cellular fecal filtrate.

In one aspect, a method achieves a remission, cure, response, or resolution rate of alopecia of at least about 10%.

In an aspect, a preparation of uncultured fecal bacteria or a fecal microbiota in a therapeutic composition comprises a donor's substantially entire and non-selected fecal microbiota, reconstituted fecal material, or synthetic fecal material.

In one aspect, the present disclosure provides for a method for treating alopecia in a subject in need thereof, the method comprising administering to the subject an oral capsule comprising a pharmaceutically active dose of a therapeutic composition comprising a preparation of uncultured fecal bacteria or a non-selected fecal microbiota.

In one aspect, the present disclosure provides for a method of treating alopecia in a subject in need thereof, the method comprising orally administering to the subject a pharmaceutically active dose of a therapeutic composition comprising a preparation of uncultured fecal bacteria or a non-selected fecal microbiota.

In one aspect, the present disclosure provides for a method of treating alopecia in a subject in need thereof, the method comprising testing said subject for the relative abundance of one or more Akkermansia or Parabacteroides species and orally administering to the subject a pharmaceutically active dose of a therapeutic composition comprising a preparation of uncultured fecal bacteria or a non-selected fecal microbiota.

In one aspect, the present disclosure provides for a method of treating alopecia in a subject in need thereof, the method comprising testing the subject for the relative abundance of one or more Clostridiales, Ruminococcaceae, Rikenellaceae, Lachnospiraceae, and Alistipes species and orally administering to the subject a pharmaceutically active dose of a therapeutic composition comprising a preparation of uncultured fecal bacteria or a non-selected fecal microbiota.

In one aspect, the present disclosure provides for a method of treating alopecia in a subject in need thereof, the method comprising testing the subject for the relative abundance of one or more Proteobacteria, Cronobacter, Bacteroides, Prevotella, and Bifidobacterium species and orally administering to the subject a pharmaceutically active dose of a therapeutic composition comprising a preparation of uncultured fecal bacteria or a non-selected fecal microbiota.

In one aspect, the present disclosure provides for a method of treating alopecia in a subject in need thereof, said method comprising orally administering to the subject a pharmaceutically active dose of a therapeutic composition comprising a preparation of uncultured fecal bacteria or a non-selected fecal microbiota, wherein the administering comprises at least 10 capsules.

### DETAILED DESCRIPTION

Unless defined otherwise herein, terms are to be understood according to conventional usage by those of ordinary skill in the relevant art.

All publications, patents, and patent applications mentioned in this specification are herein incorporated by reference to the same extent as if each individual publication, patent, or patent application was specifically and individually indicated to be incorporated by reference.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentages, density, volume and the like, is meant to encompass variations of 20%, 10%, 5%, 1%, 0.5%, or even 0.1% of the specified amount.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5%.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating alopecia.

As used herein, “microbiota,” and “flora” refer to a community of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in or prepared from a subject's feces. Typically a pharmaceutical composition described herein is prepared by incorporating such a fecal microbiota into the composition without culturing the fecal microbiota after its purification from a stool. Herein a “preparation of uncultured fecal bacteria” refers to multiple viable bacterial strains that have been harvested, extracted or purified from one or more stool samples, without culturing the strains (e.g. in culturing medium). A non-selected fecal microbiota refers to a community or mixture of fecal microbes derived from a donor's fecal sample without selection and substantially resembling microbial constituents and population structure found in such fecal sample.

In some aspects, a preparation of uncultured fecal bacteria comprises non-selected fecal bacteria. Herein “non-selected fecal bacteria” refers to a community of viable fecal bacterial strains (e.g., present in a fecal microbiota) extracted from one or more stool samples without subjecting the extracted community to environmental conditions that intentionally select for a particular type, state or taxonomic category of bacteria (e.g., by deliberate removal of certain strains of bacteria, treatment of the community with an agent such as ethanol or chloroform, or culturing). Such non-selected fecal bacteria can comprise bacterial strains in proportional content to corresponding bacterial strains in a fecal or intestinal microbiota of a normal healthy human. Steps taken to non-selectively extract a community of fecal bacteria from a stool sample can include, for example, homogenization and filtering of the stool sample to separate the fecal bacterial strains from non-cellular stool material such as fiber and rough particulate matter, as well as, for example, eukaryotic host cells and viruses. Herein typically a non-selected fecal bacterial preparation can be prepared in either aerobic or anaerobic conditions, or a combination thereof. In certain aspects, a preparation of non-selected fecal bacteria comprises all or substantially all of the bacteria of a fecal microbiota of a stool sample. In certain aspects, a preparation of non-selected fecal bacteria comprises all or substantially all of the strains of a fecal microbiota of a stool sample. In certain aspects, a preparation of non-selected fecal bacteria comprises all or substantially all of the species of a fecal microbiota of a stool sample. In certain aspects, a preparation of non-selected fecal bacteria comprises all or substantially all of the genera of a fecal microbiota of a stool sample. In certain aspects, a preparation of non-selected fecal bacteria comprises all or substantially all of the phyla of a fecal microbiota of a stool sample. Therefore, such non-selective fecal microbiota can substantially resemble microbial constituents and the bacterial community structure found in such fecal sample.

In an aspect, an uncultured bacterial community, or a preparation of uncultured fecal bacteria, comprises at least 2, 5, 10, 20, 30, 40, 50, 100, 200, 300, 400, 500, or 600 bacterial species or strains. In another aspect, an uncultured bacterial community, or a preparation of uncultured fecal bacteria, comprises between 2 and 5, 5 and 10, 10 and 20, 20 and 30, 30 and 40, 40 and 50, 50 and 60, 60 and 100, 100 and 200, 200 and 300, 300 and 400, 400 and 500, or 500 and 600 bacterial species or strains.

In an aspect, a preparation of uncultured fecal bacteria and/or non-selected fecal bacteria do not comprise an antibiotic resistant population of bacteria.

In another aspect, a preparation of uncultured fecal bacteria can involve steps that select for a particular, type, state, or taxonomic category of bacteria (e.g., by deliberate removal of certain strains of bacteria and/or treatment of the population with a selective agent such as ethanol or chloroform).

Herein a preparation of uncultured fecal bacteria is distinguished from a single, purified strain of bacteria such as a bacterial isolate. As used herein, “bacterial isolate” refers to an isolated group of substantially genetically identical bacterial cells generated by proliferation via binary fission from a single predecessor bacterial cell (e.g., by culturing the bacteria). Typically, a bacterial isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, a bacterial isolate can be grown as a pure culture of cells; in other aspects, multiple bacterial isolates can be grown simultaneously in the same vessel as a mixed culture. In the bacterial context, the term “substantially genetically identical” refers to the very high (e.g. >99.9%) genetic identity shared by different cells in uncontaminated pure compositions of bacterial isolates, owing to their proliferation from a common predecessor, but accounts for minor genetic dissimilarity between cells due to accumulations of relatively rare mutations. Generally, a bacterial isolate is synonymous with a pure culture of bacterial cells. Typically, herein a bacterial isolate consists of non-pathogenic bacteria. In an aspect, a bacterial isolate can be a probiotic, or an ingredient in a probiotic.

As used herein, the term “bacterial cocktail”, sometimes called a “bacterial consortium” or “synthetic bacterial mixture”, refers to an engineered mixture of bacteria comprising a defined consortium of multiple bacterial isolates. The term “defined consortium of multiple bacterial isolates” means that the bacterial cocktail contains two or more bacterial isolates, and that the identity of each bacterial isolate in the cocktail is known, and thus the cocktail can be consistently produced (e.g. by combining isolated bacterial strains) to have a stable composition and properties across separate batches. Herein “identity” of a bacterial isolate can refer to any characteristic of the isolate that uniquely identifies the isolate as different from one or more other bacterial isolates or bacterial strains. Examples of identifying characteristics of a bacterial isolate include nucleotide sequences such as a 16S rRNA sequence, the sequence of one or more coding or non-coding regions of a nucleic acid, and entire genome sequences, levels of gene expression, physiological or metabolic traits, or anatomical traits such as staining pattern or cell wall characteristics.

As used herein, “bacterial mixture” refers to an engineered composition comprising viable bacterial cells. In some aspects, a bacterial mixture comprises one or more non-pathogenic bacterial isolates. In some aspects, a bacterial mixture comprises a preparation of uncultured fecal bacteria. In some aspects, a bacterial mixture comprises both of one or more non-pathogenic bacterial isolates and a preparation of uncultured fecal bacteria.

As used herein, the term “relative abundance” refers to relative representation of an organism of a particular kind (e.g., a bacterial strain, species, or genus) relative to all organisms of similar nature in a certain community (e.g., a preparation of uncultured fecal bacteria or a bacterial mixture). Relative abundance is calculated by dividing the number of an organism of a particular kind by the total number of all organisms of similar nature in a certain community. In an aspect, relative abundance is measured by qPCR comparing PCR products generated with 16S primers targeting specific bacterial strains of interest against PCR products generated with universal primers targeting all 16S sequences. See e.g., Chu, N., et al., “Profiling living bacteria informs preparation of fecal microbiota transplantations.” PLoS One 12(1): 1-16 (2017). In another aspect, the relative abundance is measured based on the number of sequence reads detected via high-throughput sequencing as described in Gevers et al., “The treatment-naïve microbiomes in new-onset Crohn's disease.” Cell Host & Microbe, 15(3):382-92(2014). In an aspect, high-throughput sequencing is based on 16S rRNA gene sequencing. In another aspect, high-throughput sequencing is based on whole-genome short-gun metagenomic sequencing. Unless specified otherwise, a bacterial relative abundance mentioned herein is measured via high-throughput sequencing of 16S rRNA targeting the V4 variable region as described in Gevers et al., Cell Host & Microbe, 15(3):382-92(2014). In a further aspect, propidium monoazide (PMA) is used to differentiate between viable and dead fecal microbes as shown in Chu et al., PLoS One 12(1): 1-16 (2017).

As used herein, a “sterile fecal filtrate” or a “non-cellular fecal filtrate” refers to a liquid component of a fecal material, where the liquid component is free or substantially free of cell-based living organisms (e.g., bacteria, fungi, or their spores), but retains bacteriophages and non-cellular biological materials. Preferably, a non-cellular or sterile fecal filtrate is also free of viruses for eukaryotic host cells.

As used herein, “remission, cure, or resolution rate” refers to the percentage of patients that are cured or obtain remission or complete resolution of a condition in response to a given treatment. Quantitatively, a patient responds to a treatment positively when a symptom of the patient's alopecia is reduced, for example showing an increase in the percentage of a body surface area displaying hair.

As used herein, “response rate” refers to the percentage of patients that respond positively (e.g., reduced severity or frequency of one or more symptoms) to a given treatment.

As used herein, “eukaryotic” refers to belonging to a cell that contains a nucleus and membrane-bound organelles.

As used herein, “bacteria,” “bacterium,” and “archaea” refer to single-celled prokaryotes that lack membrane bound nuclei and lack organelles.

As used herein, “colony forming units” (cfu) refers to an estimate of the number of viable microorganism cells in a given sample.

As used herein, “viable” or “live” means possessing the ability to multiply. The viability of bacterial populations or communities can be monitored as a function of the membrane integrity of the cell. Cells with a compromised membrane are considered to be dead or dying, whereas cells with an intact membrane are considered live. For example, SYTO 9 and propidium iodide are used to stain and differentiate live and dead bacteria. See Stocks, Cytometry A. 2004 October; 61(2):189-95. Cell viability can also be evaluated via molecular viability analyses, e.g., a PCR-based approach, which can differentiate nucleic acids associated with viable cells from those associated with inactivated cells. See Cangelosi and Mescheke, Appl Environ Microbiol. 2014 October; 80(19): 5884-5891.

As used herein, “fecal bacteria” refers to bacteria that can be found in fecal matter.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). Preferred subjects are human subjects. The human subject may be a pediatric, adult or a geriatric subject. In some aspects, the terms “patient” and “subject” are used interchangeably. The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula

$$H = - {\sum }_{i = 1}^{R} {p}_{i} ln {p}_{i}$$

where H is Shannon Diversity Index, R is the total number of species in the community, and pi is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117pp.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” means that that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, a “continuous dosing schedule” refers to a dosing schedule where a therapeutic composition is administered during a treatment period without a rest period. Throughout the treatment period of a continuous dosing schedule, a therapeutic composition can be administered, for example, daily, or every other day, or every third day. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time, for example, once per day, once a week, or once in two weeks.

As used herein, “dosing interval” refers to the amount of time that elapses between multiple doses being administered to a subject.

As used herein, “adverse events (AEs)” refers to any dose that results in procedure- or microbiota-related signs or symptoms. As used herein, “serious adverse events (SAEs)” refers to any medical occurrence that at any dose: results in death or is life-threatening. As used herein “life-threatening” refers to an event in which the patient is at risk of death at the time of the event. Adverse events are graded according to a scale used by one of ordinary skill in the art (e.g., National Cancer Institute (NCI) Common Terminology Criteria for Adverse Events (CTCAE)).

In one aspect, the present disclosure provides for a method for treating a subject having a form of alopecia. As used herein, “alopecia” broadly refers to any autoimmune condition that results in loss of hair from one or more hair follicles, or the absence of production or growth of hair from one or more hair follicles of a subject.

In one aspect, the alopecia results in loss of hair or absence of production or growth of hair from a patch or patches of hair follicles of a subject. Herein a “patch” refers to a cluster of hair follicles located adjacent to one another on a body surface of a subject. In different aspects, a patch of hair follicles affected by alopecia refers to 2 or more hair follicles, 3 or more hair follicles, 5 or more hair follicles, 10 or more hair follicles, 50 or more hair follicles, 100 or more hair follicles, 200 or more hair follicles, 500 or more hair follicles, 750 or more hair follicles, 1000 or more hair follicles, or 5000 or more hair follicles, 10,000 or more hair follicles, or 20,000 or more hair follicles. In one aspect, the patch of hair follicles that has lost hair, or which does not produce or grow hair when affected by alopecia is circular. In one aspect, the patch of hair follicles affected by alopecia is coin-shaped. In one aspect, the patch of hair follicles affected by alopecia is square or rectangular. In one aspect, the patch of hair follicles affected by alopecia is triangular. In one aspect the alopecia is alopecia areata.

In one aspect, alopecia (e.g. alopecia areata) results in hair loss and/or absence of production or growth of hair from multiple patches of hair follicles on a body part or multiple body parts of a subject. In an aspect, the multiple patches of hair follicles affected by alopecia are separated by one or more hair follicles that have not lost hair or produce or grow hair. In different aspects, alopecia affects 2 or more patches of hair follicles (i.e. such that all affected hair follicles in a given patch lose hair or do not produce or grow hair), 3 or more patches of hair follicles, 3 or more patches of hair follicles, 4 or more patches of hair follicles, 5 or more patches of hair follicles, 10 or more patches of hair follicles, 15 or more patches of hair follicles, 20 or more patches of hair follicles, 25 or more patches of hair follicles, 50 or more patches of hair follicles, 100 or more patches of hair follicles, 200 or more patches of hair follicles, or 500 or more patches of hair follicles.

In one aspect, alopecia results in hair loss and/or absence of production or growth of hair from all or substantially all hair follicles on a surface of a body part of a subject. In various aspects “part of a body” or “body part” refers to a scalp, a face, a chin region, a cheek region, an eyebrow region, an eyelid, an upper lip region, a neck, a shoulder, an upper arm, a forearm, a wrist, a hand, a finger, a back, a chest, an abdomen, a hip, an upper leg, a lower leg, an ankle, a foot, a toe, a groin, a pubic region and/or genitals.

In one aspect, alopecia results in hair loss and/or absence of production or growth of hair from all or substantially all hair follicles of the scalp. In one aspect, the alopecia is alopecia totalis.

In one aspect, alopecia results in hair loss and/or absence of production or growth of hair from all or substantially all of the entire body of a subject. In one aspect, the alopecia is alopecia universalis.

In one aspect, the hair loss resulting from alopecia is radical and sudden. In one aspect the alopecia is diffuse alopecia.

In one aspect, the alopecia is temporary hair loss experienced during pregnancy or within 1 year following pregnancy. For example, the alopecia can occur in a female subject within 3-9 months after pregnancy. In an aspect, the alopecia is postpartum alopecia.

In one aspect, the present disclosure provides a method for treating alopecia in a subject in need thereof, where the method comprises administering to the subject a pharmaceutically active dose of a therapeutic composition comprising live non-pathogenic fecal bacteria.

Treatment of alopecia occurs when the patient or subject shows one or more of the following in response to administration of a therapeutic microbial composition: an increase in the percentage of a surface area of a body surface (e.g. scalp) showing the presence of hai; an increased number of hairs at a target site of a body surface; an increased width of one or more hairs at a target site of a body surface; production or growth of hair from one or more hair follicles that previously did not produce or grow hair as a result of the alopecia condition; a reduced rate of progression of overall hair loss; the lack of appearance of additional patches of a body surface lacking hair in a particular time period in a subject who shows one or more patches lacking hair on a surface of one or more body parts (in different aspects, the time period can be 1 or more days, 2 or more days, 3 or more days, 4 or more days, 5 or more days, 6 or more days, 1 or more weeks, 2 or more weeks, 3 or more weeks, 1 or more months, 2 or more months, or 6 or more months); a slowing in the rate of appearance of patches of a body surface lacking hair; a reduction in the number of patches of a body surface lacking hair; a reduction in the size of one or more patches of a body surface lacking hair (e.g. one or more patches are reduced in size by for example about 2%, about 5%, about 10%, about 15%, about 20%, about 25%, about 30%, about 35%, about 40%, about 50%, about 60%, about 70%, about 75%, about 80%, about 90%, about 95%, about 97% or about 99%); a halting or stoppage of hair loss, a maintenance of an approximately constant amount of hair on a body part affected by alopecia (e.g. resulting from approximately equal rates of hair loss and hair growth, or the absence of any hair loss or hair growth), production or growth of hair, and/or production or growth of hair to replace hair lost as a result of alopecia. Herein the term “patch of a body surface lacking hair” encompasses both loss of hair and the absence of production or growth of hair from a cluster of hair follicles located adjacent to one another on a body surface of a subject.

In another aspect, this disclosure provides use of a composition comprising live non-pathogenic fecal bacteria in the manufacture of a medication for the treatment of alopecia. In one aspect, a therapeutic composition comprises an isolated or purified population of live non-pathogenic fecal bacteria. In an aspect, a therapeutic composition comprises a preparation of uncultured fecal bacteria. In one aspect, a therapeutic composition comprises a non-selected fecal microbiota. In another aspect, a therapeutic composition comprises a non-selected and substantially complete fecal microbiota.

In an aspect, the present disclosure provides a method for treating alopecia in a subject in need thereof, where the method comprises administering to the subject a pharmaceutically active dose of a therapeutic composition comprising live non-pathogenic bacteria. In one aspect, the present disclosure provides a method for treating alopecia in a subject in need thereof, where the method comprises administering daily to the subject a pharmaceutically active dose of a therapeutic composition comprising live non-pathogenic fecal bacteria. In one aspect, a therapeutic composition is administered to an alopecia patient in need thereof at least once daily or weekly for at least two consecutive days or weeks. In one aspect, a therapeutic composition is administered at least once daily or weekly for at least 3, 4, 5, 6, 7, 8, 9, 10, 11, 12, 13, 14, or 15 consecutive days or weeks. In another aspect, a therapeutic composition is administered at least once daily or weekly for at least 1, 2, 3, 4, 5, 6, 7, 8, 9, 10, 11, or 12 consecutive days or weeks. In one aspect, a therapeutic composition is administered at least once daily or weekly for at most 4, 5, 6, 7, 8, 9, 10, 11, 12, 13, 14, 15, 16, 17, 18, 19, or 20 consecutive days or weeks. In another aspect, a therapeutic composition is administered at least once daily or weekly for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an alopecia patient in need thereof at least twice daily or weekly for at least two consecutive days or weeks. In one aspect, a therapeutic composition is administered at least twice daily or weekly for at least 3, 4, 5, 6, 7, 8, 9, 10, 11, 12, 13, 14, or 15 consecutive days or weeks. In another aspect, a therapeutic composition is administered at least twice daily or weekly for at least 1, 2, 3, 4, 5, 6, 7, 8, 9, 10, 11, or 12 consecutive days or weeks. In one aspect, a therapeutic composition is administered at least twice daily or weekly for at most 4, 5, 6, 7, 8, 9, 10, 11, 12, 13, 14, 15, 16, 17, 18, 19, or 20 consecutive days or week. In another aspect, a therapeutic composition is administered at least twice daily or weekly for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an alopecia patient in need thereof at least three times daily or weekly for at least two consecutive days or weeks. In one aspect, a therapeutic composition is administered at least three times daily or weekly for at least 3, 4, 5, 6, 7, 8, 9, 10, 11, 12, 13, 14, or 15 consecutive days or weeks. In another aspect, a therapeutic composition is administered at least three times daily or weekly for at least 1, 2, 3, 4, 5, 6, 7, 8, 9, 10, 11, or 12 consecutive days or weeks. In one aspect, a therapeutic composition is administered at least three times daily for at most 4, 5, 6, 7, 8, 9, 10, 11, 12, 13, 14, 15, 16, 17, 18, 19, or 20 consecutive days or weeks. In another aspect, a therapeutic composition is administered at least three times daily for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating alopecia in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily or weekly for at least three consecutive days or weeks. In an aspect, the present disclosure provides a method for treating alopecia in a subject in need thereof, where the method comprises administering orally to the subject a pharmaceutically active dose of a therapeutic composition comprising a preparation of uncultured fecal bacteria, where the dose is administered at a dosing schedule of at least once or twice daily or weekly for at least three consecutive days or weeks. In another aspect, a dose is administered at least once, twice, or three times daily or weekly for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating alopecia in a subject in need thereof, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, a subject being treated is a subject already with alopecia. Administration of a disclosed therapeutic composition to clinically, asymptomatic human subject who is genetically predisposed or prone to alopecia is also useful in preventing the onset of clinical symptoms of alopecia. A human subject genetically predisposed or prone to alopecia can be a human subject having a close family member or relative exhibiting or having suffered alopecia. In another aspect, a subject being treated is a subject in which alopecia is to be prevented. In another aspect, a subject being treated is predisposed or susceptible to multiples sclerosis. In another aspect, a subject being treated is a subject diagnosed as having alopecia. In one aspect, a subject being treated is a patient in need thereof. In another aspect, a patient being treated is immunocompromised. In another aspect, a patient is a patient with alopecia but otherwise healthy. In another aspect, a patient is of healthy nutrition. In yet another aspect, a patient has normal growth. In another aspect, a patient has a healthy, balanced microbiota. In another aspect, a patient is an alopecia patient in remission from alopecia. In another aspect, a patient is an alopecia patient in remission as a result of treatment with a microbial therapy described herein. In yet another aspect, a patient has no prior history of alopecia. In a further aspect, a patient has experienced at most 1, 2, or 3 alopecia recurrences in their lifetime. In another aspect, a patient has experienced at most 5 or 10 alopecia recurrences in their lifetime. Herein “alopecia recurrence” refers to the presenting of one or more symptoms of alopecia in a patient who has previously exhibited symptoms of alopecia that partially or fully resolved. In another aspect, a patient is a person at risk of alopecia. In yet another aspect, a person at risk of alopecia is a person who has underwent chemotherapy for treatment of a cancer. In yet another aspect, a person at risk of alopecia is a person with a family member who has alopecia. Herein “family member” refers to a mother, father, grandparent, great-grandparent, brother, sister, child, grandchild, great-grandchild, aunt, uncle or cousin.

In an aspect, a therapeutic composition and/or a therapeutic regimen disclosed here reduces the frequency or severity of one or more adverse events and serious adverse events associated with an alopecia treatment. In an aspect, such reduction is at least 10%, 20%, 30%, 40%, 50%, 60%, 80%, or 100% reduction.

In an aspect, a patient has a healthy gut microbiota and/or does not have dysbiosis of the gut at the time of treatment with a microbial therapy described herein. In an aspect, a patient treated with a composition described herein does not have a history of gut dysbiosis or a disorder associated with a gut dysbiosis. Examples of disorders associated with gut dysbiosis include Clostridium difficile infection, ulcerative colitis, Inflammatory Bowel Disease (IBD), Crohn's Disease and Irritable Bowel Syndrome (IBS). In an aspect, a patient treated with a composition described herein does not show symptoms of a Clostridium difficile infection at the time of treatment. In an aspect, a patient treated with a composition described herein does not show symptoms of ulcerative colitis at the time of treatment. In an aspect, a patient treated with a composition described herein does not show symptoms of Crohn's disease at the time of treatment. In an aspect, a patient treated with a composition described herein does not show symptoms of Inflammatory Bowel Disease (IBD) at the time of treatment. In an aspect, a patient treated with a composition described herein does not show symptoms of Irritable Bowel Syndrome (MS) at the time of treatment. In an aspect, a patient treated with a composition described herein does not show symptoms of diarrhea at the time of treatment.

In an aspect, a patient has dysbiosis of the gut or a disorder associated with gut dysbiosis at the time of treatment with a microbial therapy described herein. In an aspect, a patient treated with a composition described herein has a history of gut dysbiosis or a disorder associated with a gut dysbiosis. Examples of disorders associated with gut dysbiosis include Clostridium difficile infection, ulcerative colitis, Inflammatory Bowel Disease (IBD), Crohn's Disease and Irritable Bowel Syndrome (IBS). In an aspect, a patient treated with a composition described herein shows symptoms of or has been diagnosed with a Clostridium difficile infection at the time of treatment. In an aspect, a patient treated with a composition described herein shows symptoms of or has been diagnosed with ulcerative colitis at the time of treatment. In an aspect, a patient treated with a composition described herein shows symptoms of or has been diagnosed with Crohn's disease at the time of treatment. In an aspect, a patient treated with a composition described herein shows symptoms of or has been diagnosed with Inflammatory Bowel Disease (IBD) at the time of treatment. In an aspect, a patient treated with a composition described herein shows symptoms of or has been diagnosed with Irritable Bowel Syndrome (IBS) at the time of treatment.

In one aspect, a subject administered a composition described herein has been diagnosed with or shows symptoms of a mental or psychological disorder. In different aspects the mental or psychological disorder includes one or more of depression, anxiety, alexithymia and schizophrenia.

In one aspect, a subject being treated is a human patient. In one aspect, a patient is a male patient. In one aspect, a patient is a female patient. In one aspect, a patient is a premature newborn. In an aspect, a patient is a male premature newborn. In another aspect, a patient is a female premature newborn. In one aspect, a patient is a term newborn. In an aspect, a patient is a male term newborn. In another aspect, a patient is a female term newborn. In one aspect, a patient is a neonate. In one aspect, a patient is an infant. In another aspect, a patient is a male infant. In another aspect, a patient is a female infant. In one aspect, a patient is a toddler. In another aspect, a patient is a male toddler. In another aspect, a patient is a female toddler. In one aspect, a patient is a young child. In one aspect, a patient is a child. In another aspect, a patient is a male child. In another aspect, a patient is a female child. In one aspect, a patient is an adolescent. In one aspect, a patient is a pediatric patient. In another aspect, a patient is a male pediatric patient. In another aspect, a patient is a female pediatric patient. In one aspect, a patient is a geriatric patient. In another aspect, a patient is a male geriatric patient. In another aspect, a patient is a female geriatric patient. In one aspect, a patient is an adult male. In another aspect, the patient is an adult female. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In an aspect, a human patient being treated has an AA diagnosis (including AT or AU), e.g., meeting the Alopecia Areata Investigational Assessment Guideline of National Alopecia Areata Foundation (NAAF) for at least 6 months and up to 5 years in duration. In another aspect, a human patient has at least 25% scalp hair loss due to AA. In another aspect, a human patient has a SALT of Grade S2 or higher.

In an aspect, a human patient has not used systemic antibiotics, systemic antiviral, or systemic antifungal within 8 weeks prior to a fecal microbe-based treatment provided here and/or does not anticipate the use of these agents during the treatment period. In another aspect, a human patient has not received treatment for a certain period prior to a fecal microbe-based treatment provided here (e.g., at least 12 weeks for a systemic treatment, or at least 2 weeks for a topical treatment). In another aspect, a human patient has no co-existing androgenic alopecia. In another aspect, a human patient has no active skin inflammation.

In one aspect, a subject being treated is a patient on a limited diet. In another aspect, a subject being treated is a patient on a non-limited diet. In another aspect, a subject being treated is a patient on a diet comprising animal protein. In another aspect, a subject being treated is a patient on a diet comprising spicy foods. In another aspect, a subject being treated is a patient on a diet comprising high fat food.

In one aspect, a method comprises administering a therapeutic composition orally, by enema, or via rectal suppository. In one aspect, a therapeutic composition administered herein is formulated as an enteric coated (and/or acid-resistant) capsule or microcapsule, or formulated as part of or administered together with a food, a food additive, a dairy-based product, a soy-based product or a derivative thereof, a jelly, flavored liquid, ice block, ice cream, or a yogurt. In another aspect, a therapeutic composition administered herein is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is homogenized, lyophilized, pulverized and powdered. It may then be infused, dissolved such as in saline, as an enema. Alternatively the powder may be encapsulated as enteric-coated and/or acid-resistant delayed release capsules for oral administration. In an aspect, the powder may be double encapsulated with acid-resistant/delayed release capsules for oral administration. These capsules may take the form of enteric-coated and/or acid-resistant delayed release microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition administered herein is in a liquid, frozen, freeze-dried, spray-dried, foam-dried, lyophilized, or powder form. In a further aspect, a therapeutic composition administered herein is formulated as a delayed or gradual enteric release form. In another aspect, a therapeutic composition administered herein comprises an excipient, a saline, a buffer, a buffering agent, or a fluid-glucose-cellobiose agar (RGCA) media. In another aspect, a therapeutic composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a pharmaceutical composition comprises a lyophilized formulation further comprising a reducing agent. In certain embodiments, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, Glutathione, Methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a therapeutic composition comprises a cryoprotectant. As used herein, a “cryoprotectant” refers to a substance that is added to a formulation in order to protect an active ingredient during freezing. In an aspect, a cryoprotectant comprises, consists essentially of, or consists of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof. In an aspect of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehalose with 0.1% Polysorbate 80.

In another aspect, a therapeutic composition comprises a lyoprotectant. As used herein, a “lyoprotectant” refers to a substance that is added to a formulation in order to protect an active ingredient during the drying stage of a lyophilization (also known as freeze-drying) process. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. In one aspect, a lyoprotectant is a non-reducing sugar, such as trehalose or sucrose. In one aspect, a cryoprotectant or a lyoprotectant consists essentially of, or consists of, one or more substances mentioned in this paragraph and the paragraph above.

In one aspect, a cryoprotectant or a lyoprotectant comprise an intracellular agent, e.g., DMSO, Glycerol, or PEG, which penetrates inside the cell preventing the formation of ice crystals that could result in membrane rupture. In another aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In one aspect, a therapeutic composition administered herein further comprises an acid suppressant, an antacid, an H2 antagonist, a proton pump inhibitor or a combination thereof. In one aspect, a therapeutic composition administered herein substantially free of non-living matter. In another aspect, a therapeutic composition administered herein substantially free of acellular material selected from the group consisting of residual fiber, DNA, viral coat material, and non-viable material. In another aspect, a therapeutic composition administered does not comprise an acid suppressant, an antacid, an H2 antagonist, a proton pump inhibitor or a combination thereof. In yet another aspect, a therapeutic composition administered does not comprise an acid suppressant. In another aspect, a therapeutic composition administered does not comprise an antacid. In another aspect, a therapeutic composition administered does not comprise an H2 antagonist. In another aspect, a therapeutic composition administered does not comprise a proton pump inhibitor. In another aspect, a therapeutic composition administered does not comprise metoclopramide.

In one aspect, a therapeutic composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides. In another aspect, a subject is not pretreated with a prebiotic nutrient prior to treatment with a therapeutic composition. In another aspect, the therapeutic composition is not supplemented with a prebiotic nutrient.

In one aspect, a method further comprises pretreating a subject with an antibiotic composition prior to administering a therapeutic bacterial or microbiota composition. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth sub salicylate, vancomycin, streptomycin, fidaxomicin, amikacin, arbekacin, neomycin, netilmicin, paromomycin, rhodostreptomycin, tobramycin, apramycin, and a combination thereof. In another aspect, a subject is not pretreated with an antibiotic composition prior to administering a therapeutic bacterial or microbiota composition. In another aspect, the therapeutic composition is not supplemented with an antibiotic composition. In a further aspect, a method further comprises pretreating a subject with an anti-inflammatory drug prior to administration of a therapeutic bacterial or microbiota composition. In yet another aspect, a subject is not pretreated with an anti-inflammatory drug prior to administering a therapeutic bacterial or microbiota composition. In another aspect, a therapeutic bacterial or microbiota composition is not supplemented with an anti-inflammatory.

In an aspect of the present disclosure, a method further comprises administering a therapeutic bacterial or microbiota composition to a subject in need thereof, without co-administering steroids. In another aspect, the subject has not been previously treated with steroids to treat a dysbiosis. In yet another aspect, the subject is not administered a steroid at least 1, 2, 3, 4, 5, 6, 10, 15, 20, 25, 30, 35, 40, 45, or 50 weeks prior to the administering of a therapeutic composition. In a further aspect, the subject is not administered a steroid at least 1, 2, 3, 4, 5, 6, 7, 7, 9, or 10 years prior to the administering of a therapeutic composition. In yet another aspect, the subject is not treated with steroids for at least 1, 2, 3, or 4 weeks prior to or after the administering of a therapeutic composition comprising a preparation of uncultured fecal bacteria or fecal microbiota. In another aspect, the subject is not co-treated with drugs to treat conditions of dysbiosis (e.g., Crohn's disease, Ulcerative Colitis, Irritable Bowel Disease, etc.). In yet another aspect, a subject is not co-treated with thiopurines or 5-aminoscalicylate (5-ASA). In a further aspect, a subject is not co-treated with a corticosteroid, 5-ASA products, immunomodulators, anti-TNFα agents, or other medication prescribed to treat Crohn's disease, Ulcerative Colitis, Irritable Bowel Syndrome, and Irritable Bowel Disease. In another aspect, a subject is not co-treated with a drug used to treat gastrointestinal disorders.

In an aspect of the present disclosure, a method further comprises administering a therapeutic bacterial or microbiota composition to a subject in need thereof, without co-administering nonsteroidal anti-inflammatory drugs. In another aspect, the subject has not been previously treated with nonsteroidal anti-inflammatory drugs to prevent ulcerative colitis flare-ups. In yet another aspect, the subject is not administered a nonsteroidal anti-inflammatory drug at least 1, 2, 3, 4, 5, 6, 10, 15, 20, 25, 30, 35, 40, 45, or 50 weeks prior to the administering of a therapeutic composition. In a further aspect, the subject is not administered a nonsteroidal anti-inflammatory drug at least 1, 2, 3, 4, 5, 6, 7, 7, 9, or 10 years prior to the administering of a therapeutic composition. In yet another aspect, the subject is not treated with nonsteroidal anti-inflammatory drug for at least 1, 2, 3, or 4 weeks prior to or after the administering of a therapeutic composition comprising a preparation of uncultured fecal bacteria or fecal microbiota. In another aspect, the subject is not treated with mesalamine for at least 1, 2, 3, or 4 weeks prior to or after the administering of a therapeutic composition comprising a preparation of uncultured fecal bacteria or fecal microbiota.

In one aspect, a method achieves a remission, cure, response, or resolution rate of at least about 10%, 15%, 20%, 25%, 30%, 35%, 40%, 45%, 50%, 55%, 60%, 65%, 70%, 75%, 80%, 85%, 90%, 95%, 97%, or 99%. In one aspect, a treatment method achieves at least 10%, 20%, 30%, 50%, 60%, 70%, 80%, or 90% increase in coverage of an area of a body surface (e.g. scalp) with hair after 4, 8, or 12 weeks of treatment compared to baseline (e.g., immediately prior to treatment). In one aspect, a treatment method achieves at least 10%, 20%, 30%, 50%, 60%, 70%, 80%, or 90% increase in coverage of an area of a body surface (e.g. scalp) with hair in 10%, 20%, 30%, 50%, 60%, 70%, 80%, or 90% patients after 4, 8, or 12 weeks of treatment compared to baseline (e.g., immediately prior to treatment). In an aspect, the present disclosure provides a method which achieves a 10%, 20%, 30%, 40%, 50%, 60%, 70%, 80%, or 90% increase in coverage of an area of a body surface (e.g. scalp) with hair after 1, 2, 3, 4, 5, 6, 7, 8, 9, 10, 11, or 12 weeks or months of treatment. In another aspect, a method achieves a 10-20%, 20-30%, 30-40%, 40-50%, 50-60%, 60-70%, 70-80%, 80-90%, or 90-99% increase in coverage of an area of a body surface (e.g. scalp) with hair after 1, 2, 3, 4, 5, 6, 7, 8, 9, 10, 11, or 12 weeks or months of treatment.

In one aspect, a treatment method achieves at least 10%, 20%, 30%, 50%, 60%, 70%, 80%, or 90% reduction of the area of a body surface (e.g. scalp) showing the absence of hair after 4, 8, or 12 weeks of treatment compared to baseline (e.g., immediately prior to treatment). In one aspect, a treatment method achieves at least 10%, 20%, 30%, 50%, 60%, 70%, 80%, or 90% reduction of the area of a body surface (e.g. scalp) showing the absence of hair in 10%, 20%, 30%, 50%, 60%, 70%, 80%, or 90% patients after 4, 8, or 12 weeks of treatment compared to baseline (e.g., immediately prior to treatment). In an aspect, the present disclosure provides a method which achieves a 10%, 20%, 30%, 40%, 50%, 60%, 70%, 80%, or 90% reduction of the area of a body surface (e.g. scalp) showing the absence of hair after 1, 2, 3, 4, 5, 6, 7, 8, 9, 10, 11, or 12 weeks or months of treatment. In another aspect, a method achieves a 10-20%, 20-30%, 30-40%, 40-50%, 50-60%, 60-70%, 70-80%, 80-90%, or 90-99% reduction of the area of a body surface (e.g. scalp) showing the absence of hair after 1, 2, 3, 4, 5, 6, 7, 8, 9, 10, 11, or 12 weeks or months of treatment.

In one aspect, a method achieves a remission, cure, response, or resolution rate of alopecia of between about 10-20%, 20-30%, 30-40%, 40-50%, 50-60%, 60-70%, 70-80%, 80-90%, or 90-99%. In one aspect, a treatment method achieves between about 10-20%, 20-30%, 30-40%, 40-50%, 50-60%, 60-70%, 70-80%, 80-90%, or 90-99% reduction of alopecia symptoms after 4, 8, or 12 weeks of treatment compared to baseline (e.g., immediately prior to treatment). In one aspect, a treatment method achieves between about 10-20%, 20-30%, 30-40%, 40-50%, 50-60%, 60-70%, 70-80%, 80-90%, or 90-99% reduction of alopecia symptoms in about 10-20%, 20-30%, 30-40%, 40-50%, 50-60%, 60-70%, 70-80%, 80-90%, or 90-99% of patients after 4, 8, or 12 weeks of treatment compared to baseline (e.g., immediately prior to treatment). In one aspect, a treatment method achieves between about 10-20%, 20-30%, 30-40%, 40-50%, 50-60%, 60-70%, 70-80%, 80-90%, or 90-99% reduction of alopecia symptoms after 2, 4, 6, 8, 10, 12 or 14 days of treatment compared to baseline (e.g., immediately prior to treatment). In one aspect, a treatment method achieves between about 10-20%, 20-30%, 30-40%, 40-50%, 50-60%, 60-70%, 70-80%, 80-90%, or 90-99% reduction of alopecia symptoms in about 10-20%, 20-30%, 30-40%, 40-50%, 50-60%, 60-70%, 70-80%, 80-90%, or 90-99% of patients after 2, 4, 6, 8, 10, 12 or 14 days of treatment compared to baseline (e.g., immediately prior to treatment).

In an aspect the present disclosure provides for a method that prevents relapse of alopecia-related hair loss and/or lack of production or growth of hair. In an aspect, the method prevents relapse of alopecia-related hair loss and/or lack of production or growth of hair during treatment for at least 1, 2, 3, 4, 5, 6, 7, 8, 9, 10, 11, 12, 13, 14, 15, 20, 25, 30, 35, 40, 45, 50, or 55 days or weeks from the start of treatment. In another aspect, the method prevents relapse of alopecia-related hair loss and/or lack of production or growth of hair during treatment for between land 2, 2 and 4, 4 and 6, 6 and 8, 8 and 10, 10 and 20, 20 and 30, 30 and 40, or 40 and 50 days or weeks from the start of treatment. In another aspect, the method prevents relapse of alopecia-related hair loss and/or lack of production or growth of hair after treatment has stopped for at least 1, 2, 3, 4, 5, 6, 7, 8, 9, 10, 11, 12, 13, 14, 15, 20, 25, 30, 35, 40, 45, 50, or 55 days or weeks after the last treatment. In another aspect, the method prevents relapse of alopecia-related hair loss and/or lack of production or growth of hair after treatment has stopped for between land 2, 2 and 4, 4 and 6, 6 and 8, 8 and 10, 10 and 20, 20 and 30, 30 and 40, or 40 and 50 days or weeks after the last treatment.

In an aspect the present disclosure provides for a method comprising orally administering to a subject a pharmaceutically active dose of a therapeutic composition comprising live non-pathogenic fecal bacteria or a non-cellular fecal filtrate. In one aspect, the subject is deficient in one or more Akkermansia or Parabacteroides species. In another aspect, the subject in need thereof is pretreated with a probiotic prior to administration of said composition. In a further aspect, the subject in need thereof is simultaneously treated with a probiotic. In an even further aspect, the probiotic is selected from the group consisting of Akkermansia or Parabacteroides species. In another aspect, the probiotic is one or more Akkermansia or Parabacteroides species.

In an aspect, the present disclosure provides for a method comprising testing a subject for the relative abundance of one or more Akkermansia or Parabacteroides species and orally administering to the subject a pharmaceutically active dose of a therapeutic composition comprising live non-pathogenic fecal bacteria.

In an aspect, the present disclosure provides for a method comprising testing a subject for the relative abundance of one or more Clostridiales, Ruminococcaceae, Rikenellaceae, Lachnospiraceae, and Alistipes species and orally administering to said subject a pharmaceutically active dose of a therapeutic composition comprising live non-pathogenic fecal bacteria.

In an aspect, the present disclosure provides for a method comprising testing said subject for the relative abundance of one or more Proteobacteria, Cronobacter, Bacteroides, Prevotella, and Bifidobacterium species and orally administering to the subject a pharmaceutically active dose of a therapeutic composition comprising live non-pathogenic fecal bacteria.

In an aspect, the present disclosure provides for a method of diagnosing alopecia in a subject in need thereof comprising collecting stool from the subject and subjecting the stool to HPLC.

In another aspect, the present disclosure provides for a method of treating alopecia in a subject in need thereof, the method comprising orally administering to the subject a pharmaceutically active dose of a therapeutic composition comprising a preparation of uncultured fecal bacteria or a non-selected fecal microbiota. In another aspect, the method comprises administering to the subject an oral capsule comprising a pharmaceutically active dose of a therapeutic composition comprising a preparation of uncultured fecal bacteria or a non-selected fecal microbiota.

In another aspect, the present disclosure provides for a method of treating alopecia in a subject in need thereof, the method comprising testing said subject for the relative abundance of one or more Akkermansia or Parabacteroides species and orally administering to the subject a pharmaceutically active dose of a therapeutic composition comprising a preparation of uncultured fecal bacteria or a non-selected fecal microbiota. In another aspect, the method comprises testing the relative abundance of two or more Akkermansia or Parabacteroides species. In yet another aspect, the method comprises testing the relative abundance of three or more Akkermansia or Parabacteroides species. In another aspect, the method comprises testing the relative abundance of four or more Akkermansia or Parabacteroides species. In a further aspect, the method comprises testing the relative abundance of five or more Akkermansia or Parabacteroides species. In another aspect, the method comprises testing the relative abundance of six or more Akkermansia or Parabacteroides species. In another aspect, the method comprises testing the relative abundance of at least one Akkermansia or Parabacteroides species. In yet another aspect, the method comprises testing the relative abundance of at least three Akkermansia or Parabacteroides species. In another aspect, the method comprises testing the relative abundance of at least five Akkermansia or Parabacteroides species. In a further aspect, the method comprises testing the relative abundance of at least eight Akkermansia or Parabacteroides species. In another aspect, the method comprises testing the relative abundance of six or more Akkermansia or Parabacteroides species.

In a further aspect, the subject in need thereof is deficient in one or more Akkermansia or Parabacteroides species. In another aspect, the subject in need thereof is deficient in two or more Akkermansia or Parabacteroides species. In another aspect, the subject in need thereof is deficient in three or more Akkermansia or Parabacteroides species. In yet another aspect, the subject in need thereof is deficient in four or more Akkermansia or Parabacteroides species. In a further aspect, the subject in need thereof is deficient in five or more Akkermansia or Parabacteroides species. In another aspect, the subject in need thereof is deficient in 1 to 3, 3 to 5, or 5 to 10 Akkermansia or Parabacteroides species.

In one aspect, the present disclosure provides for a method of treating alopecia in a subject in need thereof, the method comprising testing the subject for the relative abundance of one or more Clostridiales, Ruminococcaceae, Rikenellaceae, Lachnospiraceae, and Alistipes species and orally administering to the subject a pharmaceutically active dose of a therapeutic composition comprising a preparation of uncultured fecal bacteria or a non-selected fecal microbiota. In another aspect, the method comprises testing the relative abundance of two or more Clostridiales, Ruminococcaceae, Rikenellaceae, Lachnospiraceae, and Alistipes species. In yet another aspect, the method comprises testing the relative abundance of three or more Clostridiales, Ruminococcaceae, Rikenellaceae, Lachnospiraceae, and Alistipes species. In another aspect, the method comprises testing the relative abundance of four or more Clostridiales, Ruminococcaceae, Rikenellaceae, Lachnospiraceae, and Alistipes species. In a further aspect, the method comprises testing the relative abundance of five or more Clostridiales, Ruminococcaceae, Rikenellaceae, Lachnospiraceae, and Alistipes species. In another aspect, the method comprises testing the relative abundance of six or more Clostridiales, Ruminococcaceae, Rikenellaceae, Lachnospiraceae, and Alistipes species. In another aspect, the method comprises testing the relative abundance of at least one Clostridiales, Ruminococcaceae, Rikenellaceae, Lachnospiraceae, and Alistipes species. In yet another aspect, the method comprises testing the relative abundance of at least three Clostridiales, Ruminococcaceae, Rikenellaceae, Lachnospiraceae, and Alistipes species. In another aspect, the method comprises testing the relative abundance of at least five Clostridiales, Ruminococcaceae, Rikenellaceae, Lachnospiraceae, and Alistipes species. In a further aspect, the method comprises testing the relative abundance of at least eight Clostridiales, Ruminococcaceae, Rikenellaceae, Lachnospiraceae, and Alistipes species. In another aspect, the method comprises testing the relative abundance of six or more Clostridiales, Ruminococcaceae, Rikenellaceae, Lachnospiraceae, and Alistipes species.

In one aspect, the subject in need thereof is deficient in one or more Clostridiales, Ruminococcaceae, Rikenellaceae, Lachnospiraceae, and Alistipes species. In another aspect, the subject in need thereof is deficient in two or more Clostridiales, Ruminococcaceae, Rikenellaceae, Lachnospiraceae, and Alistipes species. In another aspect, the subject in need thereof is deficient in three or more Clostridiales, Ruminococcaceae, Rikenellaceae, Lachnospiraceae, and Alistipes species. In yet another aspect, the subject in need thereof is deficient in four or more Clostridiales, Ruminococcaceae, Rikenellaceae, Lachnospiraceae, and Alistipes species. In a further aspect, the subject in need thereof is deficient in five or more Clostridiales, Ruminococcaceae, Rikenellaceae, Lachnospiraceae, and Alistipes species.

In one aspect, the present disclosure provides for a method of treating alopecia in a subject in need thereof, the method comprising testing the subject for the relative abundance of one or more Proteobacteria, Cronobacter, Bacteroides, Prevotella, and Bifidobacterium species and orally administering to the subject a pharmaceutically active dose of a therapeutic composition comprising a preparation of uncultured fecal bacteria or a non-selected fecal microbiota. In another aspect, the method comprises testing the relative abundance of two or more Proteobacteria, Cronobacter, Bacteroides, Prevotella, and Bifidobacterium species. In yet another aspect, the method comprises testing the relative abundance of three or more Proteobacteria, Cronobacter, Bacteroides, Prevotella, and Bifidobacterium species. In another aspect, the method comprises testing the relative abundance of four or more Proteobacteria, Cronobacter, Bacteroides, Prevotella, and Bifidobacterium species. In a further aspect, the method comprises testing the relative abundance of five or more Proteobacteria, Cronobacter, Bacteroides, Prevotella, and Bifidobacterium species. In another aspect, the method comprises testing the relative abundance of six or more Proteobacteria, Cronobacter, Bacteroides, Prevotella, and Bifidobacterium species. In another aspect, the method comprises testing the relative abundance of at least one Proteobacteria, Cronobacter, Bacteroides, Prevotella, and Bifidobacterium species. In yet another aspect, the method comprises testing the relative abundance of at least three Proteobacteria, Cronobacter, Bacteroides, Prevotella, and Bifidobacterium species. In another aspect, the method comprises testing the relative abundance of at least five Proteobacteria, Cronobacter, Bacteroides, Prevotella, and Bifidobacterium species. In a further aspect, the method comprises testing the relative abundance of at least eight Proteobacteria, Cronobacter, Bacteroides, Prevotella, and Bifidobacterium species. In another aspect, the method comprises testing the relative abundance of six or more Proteobacteria, Cronobacter, Bacteroides, Prevotella, and Bifidobacterium species.

In one aspect, the subject in need thereof is deficient in one or more Proteobacteria, Cronobacter, Bacteroides, Prevotella, and Bifidobacterium species. In another aspect, the subject in need thereof is deficient in two or more Proteobacteria, Cronobacter, Bacteroides, Prevotella, and Bifidobacterium species. In another aspect, the subject in need thereof is deficient in three or more Proteobacteria, Cronobacter, Bacteroides, Prevotella, and Bifidobacterium species. In yet another aspect, the subject in need thereof is deficient in four or more Proteobacteria, Cronobacter, Bacteroides, Prevotella, and Bifidobacterium species. In a further aspect, the subject in need thereof is deficient in five or more Proteobacteria, Cronobacter, Bacteroides, Prevotella, and Bifidobacterium species.

In one aspect, the present disclosure provides for a method of treating alopecia in a subject in need thereof, said method comprising orally administering to the subject a pharmaceutically active dose of a therapeutic composition comprising a preparation of uncultured fecal bacteria or a non-selected fecal microbiota, wherein the administering comprises at least 10 capsules.

In one aspect, the present disclosure provides for a method of treating alopecia in a subject in need thereof, comprising pretreating or co-administering the subject with one or more anti-alopecia treatments comprising medications selected from the group consisting of an immunomodulator (e.g. immunosuppressor), corticosteroid, minoxidil, diphenylcyclopropenone (DPCP), and combinations thereof. Examples of suitable corticosteroids include triamcinolone (e.g. triamcinolone acetonide or triamcinolone diacetate), betamethasone (e.g. betamethasone velarate or betamethasone dipropionate), desoximetasone, halcinonide, amcinonide, clocortolone pivalage, fluocinoloneactonide, flurandrenolide, fluocinonide, fluticasone propionate, hydrocortisone (e.g. hydrocortisone butyrate or hydrocortisone valerate), motetasone furoate, prednicarbate, alclometasone dipropionate and desonide. In certain aspects, such corticosteroids can be administered topically (e.g. as a cream, lotion or ointment), inter-lesionally (e.g. as a micronized suspension) or systemically (e.g. as a tablet or capsule or by pulse therapy). Examples of suitable immunomodulators include topical or contact immunomodulators such as squaric acid dibutylester (SADBE) and diphenylcyclopropenone and systemic immunomodulator therapy such as anti-interleukin antibody therapy (e.g. interleukin[IL]-12/IL-23p40 antibody) and administration of Janus kinase (JAK) inhibitors. In an aspect, a fecal microbe-based therapy provided here supplements one or more of the foregoing anti-alopecia treatments, and/or reduces the frequency or dosage of such one or more anti-alopecia treatments.

In one aspect, the present disclosure provides for a method of treating alopecia in a subject in need thereof, comprising pretreating or co-administering the subject with one or more anti-alopecia treatments comprising phototherapy or laser therapy, for example in combination with one or more medications. In one aspect, the phototherapy comprises an ultraviolet light therapy treatment such as PUVA. For example, a subject can undergo treatment with PUVA using either oral 8-methoxypsoralen (8-MOP) with ultraviolet A (UVA) radiation applied to a body surface affected by alopecia, or topical application of 8-MOP and UVA radiation applied to a body surface affected by alopecia.

In an aspect, the present disclosure provides for administering one or more anti-alopecia treatments (e.g. a medication, phototherapy or laser therapy) prior to administering a pharmaceutically active dose of a microbial therapeutic composition provided in the disclosure. In another aspect, the one or more anti-alopecia treatments is continued during the administering of a pharmaceutically active dose of a therapeutic composition provided in the disclosure. In another aspect, the one or more anti-alopecia treatments is discontinued during the administering of a pharmaceutically active dose of a microbial therapeutic composition provided in the disclosure. In yet another aspect, the one or more anti-alopecia treatments is administered prior to and after the completion of the administering of a pharmaceutically active dose of a microbial therapeutic composition provided in the disclosure. In a further aspect, the administering of one or more anti-alopecia treatments is decreased in dose during the administering of a pharmaceutically active dose of a microbial therapeutic composition provided in the disclosure, compared to the dose prior to the administering of a pharmaceutically active dose of a therapeutic composition. In yet another aspect, the dose of the one or more anti-alopecia treatments is decreased after the administering of a pharmaceutically active dose of a microbial therapeutic composition provided in the disclosure, compared to the dose prior to the administering of a pharmaceutically active dose of a therapeutic composition. In yet another aspect, the dose of the one or more anti-alopecia treatments is increased after the administering of a pharmaceutically active dose of a microbial therapeutic composition provided in the disclosure, compared to the dose prior to the administering of a pharmaceutically active dose of a therapeutic composition. In a further aspect, the dose of the one or more anti-alopecia treatments is decreased in dose after the administering of a pharmaceutically active dose of a microbial therapeutic composition provided in the disclosure, compared to the dose of the one or more anti-alopecia treatments during the administering of a pharmaceutically active dose of a therapeutic composition.

In an aspect, the present disclosure provides for administering a probiotic prior to administering a therapeutic composition comprising a preparation of uncultured fecal bacteria or a non-selected fecal microbiota. In another aspect, the disclosure provides for co-administering a probiotic and a therapeutic composition comprising a preparation of uncultured fecal bacteria or a non-selected fecal microbiota. In another aspect, the probiotic is selected from the group consisting of one or more Akkermansia or Parabacteroides species. In another aspect, the probiotic is selected from the group consisting of two or more Akkermansia or Parabacteroides species. In another aspect, the probiotic is selected from the group consisting of three or more Akkermansia or Parabacteroides species. In another aspect, the probiotic is selected from the group consisting of four or more Akkermansia or Parabacteroides species. In another aspect, the probiotic is selected from the group consisting of five or more Akkermansia or Parabacteroides species. In another aspect, the probiotic is selected from the group consisting of six or more Akkermansia or Parabacteroides species. In another aspect, the probiotic is selected from the group consisting of 1 to 3, 3 to 5, 5 to 8, or 8 to 10 Akkermansia or Parabacteroides species. A probiotic may be provided in a single dose or in multiple doses. When provided as a single composition, the single composition may comprise a single probiotic or a mixture of probiotics. When provided in multiple compositions, each composition may comprise a single probiotic or a mixture of probiotics.

The compositions and methods of the present invention may further comprise one or more prebiotics.

A prebiotic is a substrate that is selectively used by a host microorganism to produce a health benefit in a subject. Without wishing to be bound by theory, prebiotics are added to nutritionally supplement bacteria in the microbiome and/or in a microbial composition, e.g., to stimulate the growth or activity of one or more strains of beneficial bacteria. Additionally, the prebiotics may be added to prevent “shock” to bacterial strains subsequent to their isolation or purification, freezing, freeze-drying, spray-drying, reconstitution in solution and the like.

Examples of prebiotics include amino acids, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, tagatose, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs).

In embodiments, a prebiotic can be added (e.g., in dry or liquid forms) to a microbial composition of the present invention.

Alternately, or additionally, a prebiotic can be included (e.g., in dry or liquid forms) in a distinct pharmaceutical composition which lacks a microbial composition of the present invention.

A prebiotic may be provided to a subject before, contemporaneously with, and/or after a pharmaceutical composition comprising a microbial composition of the present invention is administered, either in a pharmaceutical composition comprising the microbial composition or in a pharmaceutical composition lacking a microbial composition.

A prebiotic may be provided in a single dose or in multiple doses. When provided as a single composition, the single composition may comprise a single prebiotic or a mixture of prebiotics. When provided in multiple compositions, each composition may comprise a single prebiotic or a mixture of prebiotics.

As examples, when multiple doses are provided, a first composition comprising a prebiotic may include one specific prebiotic, e.g., inulin, and a second composition may include a second specific prebiotic, e.g., pectin. Alternately, a first composition may include a mixture of prebiotics, e.g., inulin and pectin and a second composition may include different mixture of prebiotics, e.g., inulin and a FOS. A first composition may include a mixture of prebiotics and a second composition may include one specific prebiotic.

The amount of prebiotic provided to a subject/patient and/or included in a composition depends on the specific prebiotic, the specific bacterial strain of beneficial bacteria, and/or the disease state of the subject.

In one aspect, the present disclosure provides for determining a symptom of a subject's alopecia using one or more tests selected from the group consisting of digital photography (for determining the number and/or size of hairs at a given body surface site), and quality of life assessment tool (e.g. Alopecia Areata Symptom Impact Scale (AASIS)). In an aspect, the aforementioned tests are administered prior to administering a therapeutic composition comprising a preparation of uncultured fecal bacteria or a non-selected fecal microbiota. In an aspect, the aforementioned tests are administered after administering a therapeutic composition comprising a preparation of uncultured fecal bacteria or a non-selected fecal microbiota. In an aspect, the aforementioned tests are administered before and after administering a therapeutic composition comprising a preparation of uncultured fecal bacteria or a non-selected fecal microbiota.

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cfu. In another aspect, a pharmaceutically active therapeutic effective dose comprises at most about 10⁵, 10⁶, 10⁷, 10⁸, 10⁹, 10¹⁰, 10¹¹, 10¹², 10¹³, 10¹⁴, or 10¹⁵ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cells or spores. In another aspect, a pharmaceutically active or therapeutic effective dose comprises at most about 10⁵, 10⁶, 10⁷, 10⁸, 10⁹, 10¹⁰, 10¹¹, 10¹², 10¹³, 10¹⁴, or 10¹⁵ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter. In an aspect, a pharmaceutically active or therapeutic effective dose comprises between 10¹⁰ and 10¹² cells. In another aspect, a pharmaceutically active or therapeutic effective dose comprises between 10¹⁰ and 10¹² cells per capsule. In another aspect, a pharmaceutically active or therapeutic effective dose comprises between 10¹¹ and 10¹² cells per capsule. In a further aspect, a pharmaceutically active or therapeutic effective dose comprises between 10⁹ and 10¹² cells per capsule.

In one aspect, a therapeutic composition administered herein comprises fecal bacteria. In one aspect, a therapeutic composition administered herein comprises one or more, two or more, three or more, four or more, or five or more isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In one aspect, a therapeutic composition administered herein comprises at least one, at least two, at least three, at least four, at least five, at least six, or at least seven fecal microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, —CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, and a combination thereof.

In one aspect, a therapeutic composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a therapeutic composition administered herein comprises no viable Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Fine goldia magnus, Peptococcus G, -AU-2; Streptococcus intermedius, Ruminococcus lactaris, Ruminococcus CO Gemmiger X, Coprococcus BH, —CC; Eubacterium tenue, Eubacterium ramulus, Bacteroides clostridiiformis ssp. clostridliformis, Bacteroides coagulans, Prevotella oails, Prevotella ruminicola, Odoribacter splanchnicus, Desuifomonas pigra, Lactobacillus G, Succinivibrio A, or a combination thereof.

In one aspect, a therapeutic composition administered herein comprises a preparation of uncultured fecal bacteria or a fecal microbiota. In another aspect, the preparation of uncultured fecal bacteria or a fecal microbiota used herein involves a treatment selected from the group consisting of ethanol treatment, detergent treatment, heat treatment, irradiation, and sonication. In another aspect, the preparation of a preparation of uncultured fecal bacteria or a fecal microbiota used herein involves no treatment selected from the group consisting of ethanol treatment, detergent treatment, heat treatment, irradiation, and sonication. In one aspect, the preparation of a preparation of uncultured fecal bacteria or a fecal microbiota used herein involves a separation step selected from the group consisting of density gradients, filtration (e.g., sieves, nylon mesh), and chromatography. In another aspect, the preparation of a preparation of uncultured fecal bacteria or a fecal microbiota used herein involves no separation step selected from the group consisting of density gradients, filtration (e.g., sieves, nylon mesh), and chromatography. In another aspect, a preparation of uncultured fecal bacteria or a fecal microbiota used herein comprises a donor's entire fecal microbiota. In another aspect, a therapeutic composition administered herein comprises a preparation of uncultured fecal bacteria or a fecal microbiota substantially free of eukaryotic cells from a corresponding donor.

In another aspect, a therapeutic composition administered herein comprises a preparation of uncultured fecal bacteria or a fecal microbiota further supplemented, spiked, or enhanced with a fecal microorganism (e.g., a bacterial isolate). In one aspect, a preparation of uncultured fecal bacteria or a fecal microbiota is supplemented with a non-pathogenic (or with attenuated pathogenicity) bacterium of Clostridium, Collinsella, Dorea, Ruminococcus, Coprococcus, Prevotella, Veillonella, Bacteroides, Baccillus, or a combination thereof. In another aspect, a therapeutic composition administered herein comprises a preparation of uncultured fecal bacteria or a fecal microbiota further supplemented, spiked, or enhanced with a species of Veillonellaceae, Firmicutes, Gammaproteobacteria, Bacteroidetes, or a combination thereof. In another aspect, a therapeutic composition administered herein comprises a preparation of uncultured fecal bacteria or a fecal microbiota further supplemented with fecal bacterial spores. In one aspect, fecal bacterial spores are Clostridium spores, Bacillus spores, or both.

In an aspect, a therapeutic composition comprises a preparation of uncultured fecal bacteria or a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless.

In an aspect, a therapeutic composition provided or administered herein comprises a preparation of uncultured fecal bacteria or a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises a preparation of uncultured fecal bacteria or fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In another aspect, a therapeutic composition comprises fecal bacteria from at least 11, 12, 13, 14, 15, 16, 17, 18, 19, or 20 different families. In yet another aspect, a therapeutic composition comprises fecal bacteria from at least 21, 22, 23, 24, 25, 26, 27, 28, 29, or 30 different families. In a further aspect, a therapeutic composition comprises fecal bacteria from at least 31, 32, 33, 34, 35, 36, 37, 38, 39, or 40 different families. In another aspect, a therapeutic composition comprises fecal bacteria from at least 41, 42, 43, 44, 45, 46, 47, 48, 49, or 50 different families. In another aspect, a therapeutic composition comprises fecal bacteria from between 1 and 10, between 10 and 20, between 20 and 30, between 30 and 40, between 40 and 50 different families. In an aspect, a therapeutic composition provided or administered herein comprises a preparation of uncultured fecal bacteria or a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided or administered herein comprises a preparation of uncultured fecal bacteria or a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a therapeutic composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human. In an aspect, a therapeutic composition provided or administered herein comprises an extract of human feces where the composition is substantially odorless. In an aspect, a therapeutic composition provided or administered herein comprises fecal material or a fecal floral preparation in a lyophilized, crude, semi-purified or purified formulation.

In an aspect, a preparation of uncultured fecal bacteria or a fecal microbiota in a therapeutic composition comprises highly refined or purified fecal flora, e.g., substantially free of non-floral fecal material. In an aspect, a preparation of uncultured fecal bacteria or a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a preparation of uncultured fecal bacteria or a fecal microbiota in a therapeutic composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a preparation of uncultured fecal bacteria or a fecal microbiota in a therapeutic composition comprises a donor's substantially entire or non-selected fecal microbiota, reconstituted fecal material, or synthetic fecal material. In another aspect, a preparation of uncultured fecal bacteria or a fecal microbiota in a therapeutic composition comprises no antibiotic resistant population. In another aspect, a therapeutic composition comprises a preparation of uncultured fecal bacteria or a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a preparation of uncultured fecal bacteria or a fecal microbiota in a therapeutic composition used herein is derived from disease-screened fresh homologous feces or equivalent freeze-dried and reconstituted feces. In an aspect, a fresh homologous feces does not include an antibiotic resistant population. In another aspect, a preparation of uncultured fecal bacteria or a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a therapeutic composition used in a treatment disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, excreted or otherwise liquid components or a microbiota, e.g., biologically active molecules (BAMs), which can be antibiotics or anti-inflammatories, are preserved, retained or reconstituted in a flora extract.

In one aspect, an exemplary therapeutic composition comprises starting material from a donor from a defined donor pool, where this donor contributes a stool that is centrifuged, then filtered with very high-level filtration using e.g., either metal sieving or Millipore filters, or equivalent, to ultimately permit only cells of bacterial origin to remain, e.g., often less than about 5 micrometers diameter. After the initial centrifugation, the solid material is separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation is now taken and filtered progressively in a filtering, e.g., a Millipore filtering or equivalent systems, to end up with liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a therapeutic composition used here comprises a reconstituted fecal flora consisting essentially of a combination of a preparation of uncultured fecal bacteria or a purified fecal microbiota and a non-cellular fecal filtrate. In another aspect, a therapeutic composition used here comprises a preparation of uncultured fecal bacteria or a purified fecal microbiota supplemented with one or more non-cellular non-particulate fecal components. In one aspect, a therapeutic composition used here comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a treatment method provided here comprises the use of both fecal bacterial cells, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a fecal bacteria-based therapeutic composition is used concurrently with a fecal non-cellular filtrate-based therapeutic composition. In another aspect, a patient is treated with a first fecal non-cellular filtrate-based therapeutic composition before being given a second fecal bacteria-based therapeutic composition, or vice versa. In a further aspect, a treatment method comprises three steps: first, antibiotic pretreatment to non-selectively remove infectious pathogen(s); second, a fecal non-cellular filtrate-based treatment step to further suppress selected infectious pathogen(s); and third, giving the patient a fecal bacteria-based therapeutic composition to re-establish a functional intestinal microbiome.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of alopecia. The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a therapeutic composition comprises at least about 10⁵, 10⁶, 10⁷, 10⁸, 10⁹, 10¹⁰, 10¹¹, 10¹², or 10¹³ cfu or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cfu or total cell count.

In another aspect, a therapeutic composition comprises at least about 10⁵, 10⁶, 10⁷, 10⁸, 10⁹, 10¹⁰, 10¹¹, 10¹² or 10¹³ cells or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cells or total cell count.

In one aspect, a therapeutic composition is formulated as an oral capsule, microcapsule, tablet, or pill. In another aspect, a capsule, microcapsule, tablet, or pill is adapted for enteric delivery. In a further aspect, a capsule, microcapsule, tablet, or pill is an enteric capsule, microcapsule, tablet, or pill. In another aspect, a capsule, microcapsule, tablet, or pill comprises an enteric coating, is acid resistant, or both.

In one aspect, an exemplary therapeutic composition comprises starting material from a donor. In another aspect, an exemplary therapeutic composition comprises material from one or more healthy donors. In yet another aspect, an exemplary therapeutic composition comprises starting material from a defined donor pool. In another aspect, a donor is an alopecia patient in remission. In another aspect, a donor is an alopecia patient who was administered a preparation of uncultured fecal bacteria or a fecal microbiota as a treatment for the alopecia, and who recovered from the alopecia due to the administration. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. In another aspect, a donor is healthy. In one aspect, a human donor is a child below about 18, 15, 12, 10, 8, 6, 4, 3, 2, or 1 year old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years). In one aspect, a donor is an old individual (>85 years). In yet another aspect, a donor is a carefully screened, healthy, neurotypical human.

In an aspect, a carefully screened donor undergoes a complete medical history and physical exam. Donors are excluded if they have a risk of infectious agents. Additional exclusion criteria comprises the following:

1. Known viral infection with Hepatitis B, C or HIV
2. Known exposure to HIV or viral hepatitis at any time
3. High risk behaviors including sex for drugs or money, men who have sex with men, more than one sexual partner in the preceding 12 months, any past use of intravenous drugs or intranasal cocaine, history of incarceration.
4. Tattoo or body piercing within 12 months.
5. Travel to areas of the world where risk of traveler's diarrhea is higher than the US.
6. Current communicable disease, e.g., upper respiratory viral infection.
7. History of irritable bowel syndrome. Specific symptoms may include frequent abdominal cramps, excessive gas, bloating, abdominal distension, fecal urgency, diarrhea, constipation.
8. History of inflammatory bowel disease such as Crohn's disease, ulcerative colitits, microscopic colitis.
9. Chronic diarrhea.
10. Chronic constipation or use of laxatives.
11. History of gastrointestinal malignancy or known colon polyposis.
12. History of any abdominal surgery, e.g., gastric bypass, intestinal resection, appendectomy, cholecystectomy, etc.
13. Use of Probiotics or any other over the counter aids used by the potential donor for purpose of regulating digestion. Yogurt and kefir products are allowed if taken merely as food rather than nutritional supplements.
14. Antibiotics for any indication within the preceding 6 months.
15. Any prescribed immunosuppressive or anti-neoplastic medications.
16. Metabolic Syndrome, established or emerging. Criteria used for definition here are stricter than any established criteria. These include history of increased blood pressure, history of diabetes or glucose intolerance.
17. Known systemic autoimmunity, e.g., connective tissue disease, multiple sclerosis.
18. Known atopic diseases including asthma or eczema.
19. Chronic pain syndromes including fibromyalgia, chronic fatigue syndrome.
20. Ongoing (even if intermittent) use of any prescribed medications, including inhalers or topical creams and ointments.
21. Neurologic, neurodevelopmental, and neurodegenerative disorders including autism, Parkinson's disease.
22. General. Body mass index>26 kg/m2, central obesity defined by waste:hip ratio>0.85 (male) and >0.80 (female).
23. Blood pressure>135 mmHg systolic and >85 mmHg diastolic.
24. Skin—presence of a rash, tattoos or body piercing placed within a year, or jaundice
25. Enlarged lymph nodes.
26. Wheezing on auscultation.
27. Hepatomegaly or stigmata of liver disease.
28. Swollen or tender joints. Muscle weakness.
29. Abnormal neurologic examination.
30. Positive stool Clostridium difficile toxin B tested by PCR.
31. Positive stool cultures for any of the routine pathogens including Salmonella, Shigella, Yersinia, Campylobacter, E. coli 0157:H7.
32. Abnormal ova and parasites examination.
33. Positive Giardia, Cryptosporidium, or Helicobacter pylori antigens.
34. Positive screening for any viral illnesses, including HIV 1 and 2, Viral Hepatitis A IgM, Hepatitis surface antigen and core Ab.
35. Abnormal RPR (screen for syphilis).
36. Any abnormal liver function tests including alkaline phosphatase, aspartate aminotransaminase, alanine aminotransferase.
37. Raised serum triglycerides>150 mg/Dl
38. HDL cholesterol<40 mg/dL (males) and <50 mg/dL (females)
39. High sensitivity CRP>2.4 mg/L
40. Raised fasting plasma glucose (>100 mg/dL)

In one aspect, a subject in need thereof is administered a therapeutic composition comprising a preparation of uncultured fecal bacteria or fecal microbiota of multiple carefully screened, healthy donors. In an aspect, a subject is administered a therapeutic composition over a dosing period wherein a first dose comprises at least one therapeutic composition comprises a preparation of uncultured fecal bacteria or fecal microbiota of a single donor, and a second dose of a therapeutic composition comprises a preparation of uncultured fecal bacteria or fecal microbiota of a single donor different from the donor of the first dose. In another aspect, a first dose comprises a therapeutic composition comprising a preparation of uncultured fecal bacteria or fecal microbiota of a single donor and a second dose comprises a preparation of uncultured fecal bacteria or fecal microbiota of a donor pool. The first and second dose do not indicate the order of administration to a subject, but rather that the preparation of uncultured fecal bacteria or fecal microbiota from separate donors may be used in a non-blended form.

In another aspect, the present disclosure provides for methods for treating a subject in need thereof with capsules containing a therapeutic composition comprising a preparation of uncultured fecal bacteria or fecal microbiota from a single donor. In another aspect, a capsule comprises a therapeutic composition comprising a preparation of uncultured fecal bacteria or fecal microbiota from multiple donors. In one aspect a subject is administered two or more pills each comprising a preparation of uncultured fecal bacteria or fecal microbiota from a single but different donor.

In one aspect, the present disclosure provides for methods for treating a subject in need thereof comprising administering a therapeutic composition orally or by infusions through a colonoscope, an enema or via a nasojejunal tube. In another aspect, each administration comprises a therapeutic composition comprising a preparation of uncultured fecal bacteria or fecal microbiota of a single donor similar to or different from a prior administration in a treatment period. In another aspect, a treatment period includes administration of a first dost comprising a therapeutic composition comprising a preparation of uncultured fecal bacteria or fecal microbiota of a single donor and administration of a second dose comprising a therapeutic composition comprising a preparation of uncultured fecal bacteria or fecal microbiota of multiple donors.

This disclosure includes, but is not limited to, the following exemplary embodiments.

### Embodiment 1

A method for treating alopecia in a subject in need thereof, said method comprising administering to said subject a pharmaceutically active dose of a therapeutic composition comprising live non-pathogenic fecal bacteria.

### Embodiment 2

The method of Embodiment 1, wherein said alopecia affects the scalp of said subject.

### Embodiment 3

The method of Embodiment 1 or Embodiment 2, wherein said alopecia presents as one or more patches of hair loss.

### Embodiment 4

The method of Embodiment 3, wherein said one or more patches of hair loss comprise multiple patches.

### Embodiment 5

The method of Embodiment 3 or Embodiment 4, wherein said treating comprises reducing a size of said one or more patches of hair loss.

### Embodiment 6

The method of Embodiment 4, wherein said treating comprises reducing a number of said multiple patches.

### Embodiment 7

The method of any one of Embodiments 1-6, wherein said alopecia is alopecia areata.

### Embodiment 8

The method of Embodiment 2, wherein said alopecia results in hair loss on all or substantially all of said scalp of said subject.

### Embodiment 9

The method of Embodiment 8, wherein said alopecia is alopecia totalis.

### Embodiment 10

The method of Embodiment 1, wherein said alopecia results in hair loss on all or substantially all of the body of said subject.

### Embodiment 11

The method of Embodiment 10, wherein said alopecia is alopecia universalis.

### Embodiment 12

The method of Embodiment 1, wherein said treating comprises inducing growth of hair on a body surface of said subject.

### Embodiment 13

The method of Embodiment 12, wherein said body surface lacked hair prior to said administering.

### Embodiment 14

The method of Embodiment 12, wherein said treating further comprises inhibiting or preventing loss of said hair.

### Embodiment 15

The method of any one of Embodiments 1-14, wherein said treating comprises increasing a number of hairs on a body surface of said subject.

### Embodiment 16

The method of any one of Embodiments 1-15, wherein said treating comprises increasing a width of one or more hairs on a body surface of said subject.

### Embodiment 17

The method of any one of Embodiments 1-16, wherein said live non-pathogenic fecal bacteria comprises a non-selected fecal microbiota.

### Embodiment 18

The method of any one of Embodiments 1-17 wherein said composition comprises an isolated or purified population of said live non-pathogenic fecal bacteria.

### Embodiment 19

The method of Embodiment 1, wherein said administration is on a daily or weekly basis.

### Embodiment 20

The method of Embodiment 1, wherein said administration lasts at least 1, 2, 3, 4, 5, 6, 7, 8, 9, or 10 weeks.

### Embodiment 21

The method of Embodiment 1, wherein said dose is administered at least once daily or weekly for at least 2, 3, 4, 5, 6, 7, 8, 9, 10, 11, 12, 13, 14, or 15 consecutive days.

### Embodiment 22

The method of Embodiment 1, wherein said dose is administered at least once daily or weekly for at least 1, 2, 3, 4, 5, 6, 7, 8, 9, 10, 11, or 12 consecutive weeks.

### Embodiment 23

The method of Embodiment 1, wherein said dose is administered at least once daily or weekly for at most 3, 4, 5, 6, 7, 8, 9, 10, 11, 12, 13, 14, 15, 16, 17, 18, 19, or 20 consecutive days.

### Embodiment 24

The method of Embodiment 1, wherein said dose is administered at least once daily or weekly for at most 1, 2, 3, 4, 5, 6, 7, 8, 9, 10, 11, or 12 consecutive weeks.

### Embodiment 25

The method of Embodiment 1, wherein said dose is administered at least twice daily or weekly for at least two consecutive days.

### Embodiment 26

The method of Embodiment 25, wherein said dose is administered at least twice daily or weekly for at least 3, 4, 5, 6, 7, 8, 9, 10, 11, 12, 13, 14, or 15 consecutive days.

### Embodiment 27

The method of Embodiment 25, wherein said dose is administered at least twice daily or weekly for at least 1, 2, 3, 4, 5, 6, 7, 8, 9, 10, 11, or 12 consecutive weeks.

### Embodiment 28

The method of Embodiment 25, wherein said dose is administered at least twice daily or weekly for at most 3, 4, 5, 6, 7, 8, 9, 10, 11, 12, 13, 14, 15, 16, 17, 18, 19, or 20 consecutive days.

### Embodiment 29

The method of Embodiment 25, wherein said dose is administered at least twice daily or weekly for at most 1, 2, 3, 4, 5, 6, 7, 8, 9, 10, 11, or 12 consecutive weeks.

### Embodiment 30

The method of Embodiment 25, wherein said dose is administered at least three times daily for at least one day.

### Embodiment 31

The method of Embodiment 1, wherein said administering comprises oral administration.

### Embodiment 32

The method of Embodiment 31, wherein said oral administration comprises administering a capsule comprising said composition.

### Embodiment 33

The method of Embodiment 32, wherein said capsule is an enteric coated capsule, an acid-resistant, enteric-coated capsule, or an enteric coated microcapsule.

### Embodiment 34

The method of any one of the preceding Embodiments, wherein said method further comprises administering one or more medications selected from the group consisting of an immunomodulator, a corticosteroid, minoxidil, diphenylcyclopropenone (DPCP), and combinations thereof.

### Embodiment 35

The method of any one of the preceding Embodiments, wherein said method further comprises administering a phototherapy or laser therapy.

### Embodiment 36

The method of Embodiment 35, wherein said phototherapy is PUVA.

### Embodiment 37

A method of treating alopecia in a subject in need thereof, said method comprising orally administering to said subject a pharmaceutically active dose of a therapeutic composition comprising a non-selected fecal microbiota.

### Embodiment 38

The method of Embodiment 37, wherein said composition is in a liquid, frozen, freeze-dried, spray-dried, foam-dried, lyophilized, or powder form.

### Embodiment 39

The method of Embodiment 37, wherein said composition is in a capsule.

### Embodiment 40

The method of Embodiment 37, wherein said subject is pretreated with an antibiotic prior to administration of said composition.

### Embodiment 41

The method of Embodiment 40, wherein said antibiotic is selected from the group consisting of rifabutin, clarithromycin, clofazimine, vancomycin, rifampicin, nitroimidazole, chloramphenicol, and a combination thereof.

### Embodiment 42

The method of Embodiment 40, wherein said antibiotic is selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.

### Embodiment 43

The method of Embodiment 37, wherein said administering comprises an induction treatment and a maintenance treatment.

### Embodiment 44

The method of Embodiment 43, wherein said induction treatment comprises administering a greater number of capsules per day than said maintenance treatment.

### Embodiment 45

The method of Embodiment 37, wherein said subject in need thereof is deficient in one or more Akkermansia or Parabacteroides species.

### Embodiment 46

The method of Embodiment 37, wherein said subject in need thereof is pretreated with a probiotic prior to administration of said composition.

### Embodiment 47

The method of Embodiment 37, wherein said administering further comprises co-administering a probiotic.

### Embodiment 48

The method of Embodiment 47 or 48, wherein said probiotic is selected from the group consisting of Akkermansia or Parabacteroides species.

### Embodiment 49

The method of Embodiment 37, wherein said administration is on a daily or weekly basis.

### Embodiment 50

The method of Embodiment 37, wherein said administration lasts at least 1, 2, 3, 4, 5, 6, 7, 8, 9, or 10 weeks.

### Embodiment 51

The method of Embodiment 37, wherein said dose is administered at least once daily or weekly for at least 2, 3, 4, 5, 6, 7, 8, 9, 10, 11, 12, 13, 14, or 15 consecutive days.

### Embodiment 52

The method of Embodiment 37, wherein said dose is administered at least once daily or weekly for at least 1, 2, 3, 4, 5, 6, 7, 8, 9, 10, 11, or 12 consecutive weeks.

### Embodiment 53

The method of Embodiment 37, wherein said dose is administered at least once daily or weekly for at most 3, 4, 5, 6, 7, 8, 9, 10, 11, 12, 13, 14, 15, 16, 17, 18, 19, or 20 consecutive days.

### Embodiment 54

A method of treating alopecia in a subject in need thereof, said method comprising testing said subject for the relative abundance of one or more Akkermansia or Parabacteroides species and orally administering to said subject a pharmaceutically active dose of a therapeutic composition comprising a non-selected fecal microbiota.

### Embodiment 55

A method of treating alopecia in a subject in need thereof, said method comprising testing said subject for the relative abundance of one or more Clostridiales, Ruminococcaceae, Rikenellaceae, Lachnospiraceae, and Alistipes species and orally administering to said subject a pharmaceutically active dose of a therapeutic composition comprising a non-selected fecal microbiota.

### Embodiment 56

A method of treating alopecia in a subject in need thereof, said method comprising testing said subject for the relative abundance of one or more Proteobacteria, Cronobacter, Bacteroides, Prevotella, and Bifidobacterium species and orally administering to said subject a pharmaceutically active dose of a therapeutic composition comprising a non-selected fecal microbiota.

### Embodiment 57

A method of treating alopecia in a subject in need thereof, said method comprising orally administering to said subject a pharmaceutically active dose of a therapeutic composition comprising a non-selected fecal microbiota, wherein said administering comprises at least 10 capsules.

### Embodiment 58

A method of treating alopecia in a subject in need thereof, said method comprising administering to said subject a pharmaceutically active dose of a therapeutic composition comprising a non-selected fecal microbiota, wherein said subject is free of gut dysbiosis during said treating.

### Embodiment 59

A method of treating alopecia in a subject in need thereof, said method comprising administering to said subject a pharmaceutically active dose of a therapeutic composition comprising a non-selected fecal microbiota, wherein said subject does not exhibit signs of malnutrition or growth retardation.

### Embodiment 60

A method of treating alopecia in a subject in need thereof, said method comprising administering to said subject an antibiotic and a pharmaceutically active dose of a therapeutic composition comprising a non-selected fecal microbiota.

### Embodiment 61

The method of Embodiment 60, wherein said antibiotic is Vancoymcin or Rifaximin.

### Embodiment 62

A method of treating alopecia in a subject in need thereof, said method comprising administering to said subject a pharmaceutically active dose of a first and second therapeutic composition, wherein said first and second therapeutic composition each comprise a non-selected fecal microbiota.

### Embodiment 63

The method of Embodiment 62, wherein at least one of said first and second therapeutic composition comprises an oral capsule.

### Embodiment 64

The method of Embodiment 62, wherein said first therapeutic composition comprises a non-selected fecal microbiota from a first donor and said second therapeutic composition comprises a non-selected fecal microbiota from a second donor.

### Embodiment 65

The method of Embodiment 62, wherein said first and second therapeutic compositions are administered to said subject on different days.

### Embodiment 66

The method of Embodiment 65, wherein at least one of said first and second therapeutic composition comprises a non-selected fecal microbiota from a donor pool.

### Embodiment 67

The method of Embodiment 1, wherein said subject does not show symptoms of a gut dysbiosis.

### Embodiment 68

The method of Embodiment 67, wherein said subject does not show symptoms of a disorder selected from Crohn's disease, Inflammatory Bowel Disease (IBD), Irritable Bowel Syndrome (IBS), a Clostridium difficile infection, ulcerative colitis, diarrhea, and a combination thereof.

### Embodiment 69

A method for reducing or eliminating steroid use of an alopecia treatment regimen in a subject in need thereof, said method comprising administering to said subject a pharmaceutically active dose of a therapeutic composition comprising viable non-pathogenic fecal bacteria.

### Embodiment 70

A method for reducing or eliminating an immunomodulator, a corticosteroid, minoxidil, or diphenylcyclopropenone (DPCP) from an alopecia treatment regimen in a subject in need thereof, said method comprising administering to said subject a pharmaceutically active dose of a therapeutic composition comprising viable non-pathogenic fecal bacteria.

### Embodiment 71

A method for treating alopecia in a subject in need thereof, said method comprising administering to said subject a pharmaceutically active dose of a non-selected fecal microbiota following a treatment regimen, wherein said treatment regimen comprises a loading or treatment phase followed by a maintenance phase having a lower dosage or frequency relative to said loading or treatment phase.

### Embodiment 72

The method of Embodiment 71, wherein said loading or treatment phase lasts for no more than 6, 5, 4, 3, 2, or 1 week.

### Embodiment 73

The method of Embodiment 71, wherein said loading or treatment phase lasts for more than 6, 5, 4, 3, 2, or 1 week.

### Embodiment 74

The method of Embodiment 71, wherein said loading or treatment phase lasts for about 6, 5, 4, 3, 2, or 1 week.

### Embodiment 75

The method of any one of Embodiments 71 to 74, wherein said maintenance phase lasts for no more than 24, 20, 16, 15, 14, 13, 12, 10, 8, 6, 4, or 2 weeks.

### Embodiment 76

The method of any one of Embodiments 71 to 74, wherein said maintenance phase lasts for more than 24, 20, 16, 15, 14, 13, 12, 10, 8, 6, 4, or 2 weeks.

### Embodiment 77

The method of any one of Embodiments 71 to 74, wherein said maintenance phase lasts for about 24, 20, 16, 15, 14, 13, 12, 10, 8, 6, 4, or 2 weeks.

### EXAMPLES

### Example 1. Preparation of Fecal Microbiota

Fecal microbiota is prepared essentially according to protocols published in US2014/0147417 or WO2014/152484. Summarized below is an exemplary protocol.

Potential fecal microbiota donors are screened according to a list of criteria used to exclude unsuitable donors. Potential fecal microbiota donors are excluded if they have received antibiotics, laxatives, diet pills, immunomodulators or chemotherapy in the preceding three months. Potential fecal microbiota donors are excluded if they have a history of all known infectious diseases, morbid obesity, diabetes, irritable bowel syndrome, inflammatory bowel disease, chronic diarrhea, constipation, colorectal polyps or cancer, a compromised immune system, metabolic syndromes, chronic fatigue syndrome, major GI surgery, or other diseases or conditions potentially associated with specific changes in fecal microbiota. Potential fecal microbiota donors are excluded if they exhibit positive laboratory tests for C-reactive protein, erythrocyte sedimentation rate, hepatitis A, hepatitis B, hepatitis C, human immunodeficiency virus, human T-lymphotropic virus, or syphilis. Potential fecal microbiota donors are excluded if they exhibit a positive test for stool ova, parasites, and/or viruses. Potential fecal microbiota donors are excluded if they engage in high-risk sexual behaviors, have been incarcerated, or received any tattoos or body piercings in areas that have had disease epidemics within the past three months.

Donor fecal material (fresh feces) is collected in a sterilized container, and then it is transferred to a blender. Approximately 500-1000 mL 0.9% saline solution is added to the blender and thoroughly mixed with the fecal sample. The resulting suspension is filtered at least 4 times through strainers prior to collecting a final suspension. The final suspension is centrifuged in 50 mL tubes at 1200×g for 3 minutes. The supernatant is discarded and the pellet is gently resuspended in approximately 50 mL of sterile 0.9% saline solution. The centrifugation and resuspension steps are repeated 2 to 4 additional times. Upon the final centrifugation, the supernatant is discarded. If the fecal microbiota is to be used immediately, the resultant pellet is resuspended in 1.5-volumes of 0.9% saline solution by gently mixing. If the fecal microbiota is to be stored, the resultant pellet is resuspended in 10% sterile glycerol and stored at −80 degrees Centigrade. If fecal microbiota are frozen, they are warmed to room temperature prior to administration to a patient.

### Example 2. Fecal Microbe-Based Therapy of Alopecia

An open-label, single centre, two-arm study comprising 12 male or female participants (inclusive) is conducted to investigate the safety and efficacy of a fecal microbe-based therapy for Alopecia Areata (AA), including alopecia totalis (AT) or alopecia universalis (AU). Participants who provide informed consent and meet all the inclusion criteria and none of the exclusion criteria is enrolled in the study.

In the first treatment arm (Group A or Schedule A, Table 1), the initial 6 enrolled participants receive a colonoscopy with a liquid loading dose of a non-selected fecal microbiota (FMT transplanted by colonic enema) followed by lyophilised non-selected fecal microbiota (LNSFM) capsules administered orally during Weeks 1 through 12 (6 LNSFM capsules/day), with an additional 40 weeks of follow-up post-treatment.

In the second treatment arm (Group B or Schedule B, Table 2), 6 participants will receive a 12-week course of LNSFM capsules administered orally (including a two week loading dose of 18 LNSFM capsule per day followed by 10 weeks of 6 LNSFM capsules per day), with an additional 40 weeks of follow-up post-treatment.

TABLE 1
Treatment schedule A: Patients are provided with
504 capsules which are stored in a fridge until use.

| Week of | | | |
|---|---|---|---|
| Treatment | Morning | Evening | Capsules/Day |
| Week 1-12 | 3 | 3 | 6 |

TABLE 2
Treatment schedule B Patients are provided with
652 capsules which are stored in a fridge until use.

| Week of | | | | |
|---|---|---|---|---|
| Treatment | Morning | Midday | Evening | Capsules/Day |
| Week 1-2 | 6 | 6 | 6 | 18 |
| Week 3-12 | 3 | — | 3 | 6 |

The primary efficacy endpoint is the Severity of Alopecia Tool (SALT) Score which is to be collected according to the Schedule of Assessments and analysed in the per-protocol (PP) population. The secondary endpoints that will be measured at the designated time points according to the Schedule of Assessments and analysed in the PP population are: (a) the number and width of terminal hairs in the target site using digital photography; (b) Quality of life (QoL) as assessed by the Alopecia Areata Symptom Impact Scale (AASIS); and (c) microbiome sequencing from stool samples.

The primary efficacy endpoint of change from baseline in SALT score is summarised using descriptive statistics. The primary endpoint analyses are performed on the per-protocol (PP) population.

Change from baseline in the secondary endpoints (number and width of terminal hairs in the target site using digital photography; QoL assessed by AASIS; microbiome sequencing) is summarised using descriptive statistics. The secondary endpoint analyses are performed on the PP population.

The following Inclusion Criteria are used in the study:
1. Males or females aged 18 to 75 years (inclusive)
2. Diagnosis of AA including AT or AU, by a health care professional, (i.e., meet the Alopecia Areata Investigational Assessment Guideline of National Alopecia Areata Foundation (NAAF) for at least 6 months and up to 5 years in duration)
3. Have at least 25% scalp hair loss due to AA
4. Have a SALT of Grade S2 or higher
5. Provide written informed consent to participate as shown by a signature on the consent form.

The following Exclusion Criteria are used in the study:
1. Have received FMT for any condition within one year prior to screening
2. Have used systemic antibiotics, systemic antiviral, or systemic antifungal within 8 weeks prior to screening and/or anticipate the use of these agents during the treatment period
3. Have received treatment prior to screening for AA (12 weeks systemic, or 2 weeks topical) or anticipate to start systemic and/or topical treatment for AA before the end of the study
4. Have co-existing androgenic alopecia
5. Show clinical evidence of active skin inflammation
6. Have a history of significant psychiatric diseases, in the opinion of the Principal Investigator
7. If female, are pregnant or lactating
8. Have any clinically significant conditions that may jeopardize the safety or rights of the participants, or may confound the results of the study, in the opinion of the Principal Investigator
9. Have been involved in an experimental drug study within 4 weeks prior to screening.

The LNSFM capsules product is derived from healthy human intestinal microbiota, which is processed through specialised steps for administration to participants. The LNSFM capsules product is encapsulated for oral administration.

Patients in Group A receive one dose of FMT infusion via colonoscopy followed by a standard, lyophilized FMT in an encapsulated form. Each capsule contains approximately 10¹¹ live bacterial cells and is provided 2×3/day total 6/day for twelve weeks, morning and evening via oral administration (i.e. 504 doses).

Group B only receives lyophilized FMT capsules. Participants are provided with 6×3/day total 18/day for two weeks, morning, midday and evening via oral administration (i.e. 252 doses) followed by maintenance with 6 capsules/day for the next 10 weeks (i.e. 420 doses). Participants in this group also complete the colonoscopy bowel preparation (but will not undergo a colonoscopy) prior to commencing the capsules.

Participants are required to take an oral proton-pump inhibitor medication (Somac, 40 mg) 2-3 h prior to capsule intake, to promote the viable passage of LNSFM through the stomach. The LNSFM are self-administered by the participant at home.

The concomitant use of the following medications is prohibited during the study: topical and/or oral corticosteroids; systemic antibiotic, antiviral or antifungal agents; and any treatment for the management of AA.

The Severity of Alopecia Tool (SALT) Scores are measured at the time points indicated in the Schedule of Assessments (Table 3). The SALT assesses of the degree of hair loss measured as a percentage of the scalp surface area affected (Olsen E A, Canfield D. SALT II: A new take on the Severity of Alopecia Tool (SALT) for determining percentage scalp hair loss. J Am Acad Dermatol. 2016; 75(6)1268-1270).

Participants also complete the Alopecia Areata Symptom Impact Scale (AASIS) Quality of Life QoL assessment at the time points indicated in the Schedule of Assessments. The AASIS is a validated, disease-specific measure of AA symptoms and their impact on daily functioning (Mendoza et al, The Utility and Validity of the Alopecia Areata Symptom Impact Scale in Measuring Disease-Related Symptoms and their Effect on Functioning. J Investig Dermatol Symp Proc. 2018 January; 19(1):S41-S46).

At-home stool samples (for microbiome sequencing) are collected according to the Schedule of Assessments. Instructions for collection, handling, storage, and transportation/shipping are provided to participants.

Blood samples are collected according to the Schedule of Assessments and evaluated for disease biomarkers. The following tests are conducted to determine these biomarkers' association with AA: (a) Immune-Analysis: cluster of differentiation (CD)3+, CD8+ T cells, CD11C(+) dendritic cells and CD1a(+) Langerhans cells; and (b) Quantitative reverse transcriptase polymerase chain reaction (qRT-PCR): (i) Inflammatory markers (IL-2, interleukin-2 receptor alpha chain [IL-2RA], IL-15); (ii) Immune Axes: T-helper 1 [Th1] (C-X-C motif chemokine ligand [CXCL]10 and CXCL9), Th1 (IL-13, [C-C motif chemokine ligand [CCL]17 and CCL18); and (iii) Cytokine pathways: IL-12/IL-23p40 and IL-32.

Safety is assessed through the incidence of adverse events (AEs), laboratory tests (including haematology, clinical chemistry, liver function and renal functions), and findings in physical examinations. Adverse events are assessed at each visit throughout the study by open general questioning from the investigator or study staff and entered into the CRF. The use of other medications during the study is also noted along with the reason for use in order to capture other potential adverse effects.

As various modifications could be made in the constructions and methods herein described and illustrated without departing from the scope of the disclosure, it is intended that all matter contained in the foregoing description shall be interpreted as illustrative rather than limiting. The breadth and scope of the present disclosure should not be limited by any of the above-described exemplary embodiments, but should be defined only in accordance with the following claims appended hereto and their equivalents. All patent and non-patent documents cited in this specification are incorporated herein by reference in their entireties.

TABLE 3
Schedule of assessments.

| | | | Telephone Visit | | | |
|---|---|---|---|---|---|---|
| | | | End-of-Loading | | Initial | Final |
| | Screening | Baseline | Dosing | End-of-Treatment | Follow-Up | Follow-Up |
| Assessments | Visit 1 | Visit 2 | Visit 3 | Visit 4 | Visit 5 | Visit 6 |
| and Recordings | Day 0 | Day 1 | Day 14 ± 7 | Day 84 ± 7 | Day 224 ± 7 | Day 365 ± 7 |
| Participant Information | X | | | | | |
| Informed Consent | X | | | | | |
| Inclusion/Exclusion Criteria | X | X | | | | |
| Demography | X | | | | | |
| Medical History^{a} | X | | | | | |
| Physical Examination | X | | | X | | |
| Concomitant Medications | X | X | X | X | X | X |
| AASIS QoL Questionnaire | | X | | X | X | X |
| Stool Collection Kit (for microbiome | X | X | | X | X | |
| sequencing) Dispensed | | | | | | |
| Telephone Reminder to Collect Stool | | | X | | | |
| Sample | | | | | | |
| Prior Visit Stool Collection Kit Returned | | X | | X | X | X |
| Colonoscopy With Liquid FMT Dose^{b} | | X | | | | |
| Dispense LNSFM Capsules | | X | | | | |
| Adverse Events | | X | X | X | X | X |
| Clinical Laboratory Evaluation^{c} | X | | | X | X^{d} | X^{d} |
| SALT Assessment | X | | | X | X | X |
| Clinical Photography and Dermoscopy | | X | | X | X | X |

AASIS = Alopecia Areata Impact Scale, FMT = faecal microbiota transplantation, LNSFM = lyophilised non-selected fecal microbiota, QoL = quality of life; SALT = Severity of Alopecia Tool; X = assessment made at this timepoint.
^{a}Medical history including alopecia history and confirmation of diagnosis by National Alopecia Areata Foundation (NAAF) criteria.
^{b}Colonoscopy with liquid FMT dose by colonic enema, followed by oral maintenance dose with LNSFM capsules performed for the initial 6 participants only. The final 6 participants will receive both loading and maintenance FMT doses orally via LNSFM capsules.
^{c}Clinical laboratory evaluation includes: standard haematology, full biochemistry, liver and renal functions, serum trace elements [at screening and end-of-treatment only], alopecia areata disease biomarkers, and pregnancy test [females only])
^{d}Blood collection at Week 32 and Week 52 will be for alopecia areata disease biomarkers only.

1. A method for treating alopecia in a subject in need thereof, said method comprising administering to said subject a pharmaceutically active dose of a therapeutic composition comprising live non-pathogenic fecal bacteria.

2. The method of claim 1, wherein said alopecia affects the scalp of said subject.

3. The method of claim 1, wherein said alopecia presents as one or more patches of hair loss.

4. The method of claim 3, wherein said one or more patches of hair loss comprise multiple patches.

5. The method of claim 3, wherein said treating comprises reducing a size of said one or more patches of hair loss.

6. The method of claim 4, wherein said treating comprises reducing a number of said multiple patches.

7. The method of claim 1, wherein said alopecia is alopecia areata.

8. The method of claim 2, wherein said alopecia results in hair loss on all or substantially all of said scalp of said subject.

9. The method of claim 8, wherein said alopecia is alopecia totalis.

10. The method of claim 1, wherein said alopecia results in hair loss on all or substantially all of the body of said subject.

11. The method of claim 10, wherein said alopecia is alopecia universalis.

12. The method of claim 1, wherein said treating comprises inducing growth of hair on a body surface of said subject.

13. The method of claim 1, wherein said treating comprises increasing a number of hairs on a body surface of said subject.

14. The method of claim 1, wherein said treating comprises increasing a width of one or more hairs on a body surface of said subject.

15. The method of claim 1, wherein said live non-pathogenic fecal bacteria comprises a preparation of uncultured fecal bacteria.

16. The method of claim 1, wherein said composition comprises an isolated or purified population of said live non-pathogenic fecal bacteria.

17. The method of claim 1, wherein said administration is on a daily or weekly basis.

18. The method of claim 1, wherein said administration lasts at least 1, 2, 3, 4, 5, 6, 7, 8, 9, or 10 weeks.

19. The method of claim 1, wherein said administering comprises oral administration.

20. The method of claim 1, wherein said method further comprises administering one or more medications selected from the group consisting of an immunomodulator, a corticosteroid, minoxidil, diphenylcyclopropenone (DPCP), and combinations thereof.
